SEQUENCE LISTING (48 sequences)

SEQ ID NO: 1 - DNA, 2136 bp, homo sapiens
ggacgttggggagggggctgtgccgggcggagagaactcagcgaggattctgagaatggtagataacgcagatcatctctggaaaggatattgatccgcctcatgtaaagtatgcttagttcctttccagtggttttgctggaaaccatgtctcattatacagatgaacccagatttaccatagagcagatagatctgcttcagcgacttcggcgtactggaatgactaaacatgaaattctccatgccttggaaactttggaccgtcttgatcaagagcatagtgacaagtttggaagaaggtccagctatggaggaagttcatatgggaatagtactaacaatgtcccagcatcttcctctacagctacagcttccacacagacgcagcattcgggaatgtccccgtcacctagcaacagttatgacacttccccacagccttgcactaccaatcaaaatgggagggagaataatgagcgattatctacatccaatggaaagatgtcaccaactcgctaccatgcaaacagcatgggtcagaggtcatacagttttgaagcctcagaagaggacctagatgtagatgataaagtggaagaattaatgaggagggacagcagtgtgataaaagaggaaatcaaagcctttcttgccaatcggaggatttcccaagcagttgttgcacaggtaacaggtatcagtcagagccggatctctcattggctgttgcagcagggatcagacctgagtgaacagaagaaaagagcattttaccgatggtatcaacttgagaagacaaaccctggcgctacactaagtatgagaccagcccccattccaatagaggaccctgaatggagacaaacgcctcccccagtctctgccacatctggtactttccgactgcgacgagggagtcgatttacctggagaaaggagtgcctggctgttatggaaagttacttcaatgagaatcaatacccagatgaagcaaagagggaagaaattgcaaacgcttgcaatgcagttatacagaagccaggcaaaaagctgtcagatctggaaagagttacctccctgaaagtatataattggtttgctaacagaaggaaggagatcaagaggagagccaatattgaagcagcaatcctggagagtcatgggatagatgtgcagagtccaggaggccactcaaacagtgatgatgtcgacgggaatgactactctgagcaggatgacagtacgagccatagtgaccaccaagaccccatctcattagctgtggaaatggcagcagtcaaccacactatcttggcattggcccgacaaggagccaacgaaatcaagacagaggccctggatgatgactgatcagggaggttaaacatgacaagttaacttagtttagacgtagcaccttagcagactttcctcggtccttaacatgtgttcttacagtataacttgcagtttcttgtatgtcaggtagctgttagggtcttgttctgtgaagatggcatggtgccctcagcctttgcatatactctctcagtattaactcccagtaaataataaccaaccaaccaaccaaacttccctctcccagcccccgaggctagaaaatcttgctgctccgtcttagcattccaagaaagtgcttccaggtatttagatagccctcagttctcaaatattagactacgtgtaaaatcttgggtacctttagattcttgtaacactagtctgtactcccttttccttccccaagactgataggatgcaagctgaggtcgtggcacaggaatgacagacaccatttggggagtatccacagagtcaaaggaacactagaatccccacctcagcgtgaggataattgatttccagctgcaataagccgtgcctcattatagccacactgtggctagattatacttctttgggtgctgtgctaagaatgtcaatggaaaaagccgatctcagattttgtttgaagttaacatgcctgacacagacatcctttcctctcacaagctgtgtgacttagtagataaaatactgccttctgcctttgggaccatgattaaaaacaaagacaaaaaccaaaagtcattaaaaaaaaaaaaaaaaaaa

SEQ ID NO: 2 - protein, 420 aa, Homo sapiens
MLSSFPVVLLETMSHYTDEPRFTIEQIDLLQRLRRTGMTKHEILHALETLDRLDQEHSDKFGRRSSYGGSSYGNSTNNVPASSSTATASTQTQHSGMSPSPSNSYDTSPQPCTTNQNGRENNERLSTSNGKMSPTRYHANSMGQRSYSFEASEEDLDVDDKVEELMRRDSSVIKEEIKAFLANRRISQAVVAQVTGISQSRISHWLLQQGSDLSEQKKRAFYRWYQLEKTNPGATLSMRPAPIPIEDPEWRQTPPPVSATSGTFRLRRGSRFTWRKECLAVMESYFNENQYPDEAKREEIANACNAVIQKPGKKLSDLERVTSLKVYNWFANRRKEIKRRANIEAAILESHGIDVQSPGGHSNSDDVDGNDYSEQDDSTSHSDHQDPISLAVEMAAVNHTILALARQGANEIKTEALDDD

SEQ ID NO: 3 - protein, 419 aa, Mus musculus
MLSSFPVVLLETMSHYTDEPRFTIEQIDLLQRLRRTGMTKHEILHALETLDRLDQEHSDKFGRRSSYGGSSYGNSTNNVPASSSTATASTQTQHSGMSPSPSNSYDTSPLPCTTNQNGRENNDRLSTSNGKMSPSRYHANSMGQRSYSFEASEEDLDVDDKVEELMRRDSSVIKEEIKAFLANRRISQAVVAQVTGISQSRISHWLLQQGSDLSEQKKRAFYRWYQLEKTNPGATLSMRPAPIPIEDPEWRQTPPPVSATPGTFRLRRGSRFTWRKECLAVMESYFNENQYPDEAKREEIANACNAVIQKPGKKLSDLERVTSLKVYNWFANRRKEIKRRANIAAILESHGIDVQSPGGHSNSDDVDGNDYSEQDDSTSHSDHQDPISLAVEMAAVNHTILALARQGANEIKTEALDDD

SEQ ID NO: 4 - protein, 405 aa, Xenopus laevis
MSHYTDEPRFTIKQIDLLQRLRRTGMTKNAILHALETLERLDQEHSDKFGRRSTYGTSSYSTNNVPASSSTATASTQTLHGGMSPSPSNSCDTSPQPCTTNQNGRESSERVATSNGKLSPTRYHANSIGPRSYSFEASEEDLDVDDKVEELMRRDSSVIKEEIKAFLANRRISQAVVAQVTGISQSRISHWLLQQGSDLSEQKKRAFYRWYQLEKTNPGATLSMRPAPIPIDTDAEWRQTPPPVTATSGTFRLRRGSRFTWRKECLAVMESYFNENQYPDEAKREEIANACNAVIQKPGKKLSDLERVTSLKVYNWFANRRKEIKRRANIAILESHGIDVQSPGGHSNSDDVDGNDYSEQDDSTSHSDHQDPISLAVEMAAVNHTILALARQGTGEIKTEALDDD

SEQ ID NO: 5 - protein, 454 aa, Gallus gallus
MLRTFPVVLLETMSHYTDEPRFTIEQIDLLQRLRRTGMTRHEILHALETLDRLDQEHSDKFGRRSSYGGGSYSNSTNNVPASSSTATASTQTQHSGMSPSPSNSYDTSPQPCTTNQNGRESNERLSAFNGKMSPTRYPLANSLAQRSYSFEASEEDLDIDDKVEELMRRDSSVIKEEIKAFLANRRISQAVVAQVTGISQSRISHWLLQQGSDLSEQKKRAFYRWYQLEKTNPGATLSMRPAPIPIEEPEWRQTPPPVTATSGTFRLRRGSRFTWRKECLAVMESYFNENQYPDEAKREEIANACNAVIQKPGKKLSDLERVTSLKVYNWFANRRKEIKRRANIAAILESHGIDVQSPGGHSNSDDVDGNDYSEQDTWQVRNGEEEGRCSEGGREAEKVEEDRRICSKQDDSTSHSDHQDPISLAVEMAAVNHTILALARQGANEIKTEALDDD

SEQ ID NO: 6 - protein, 422 aa, Rattus norvegicus
MELLELILQLKPNRGSVFPSLLTREECECTDSEFHVCPYSVSLLETMSHYTDEPRFTIEQIDLLQRLRRTGMTKHEILHALETLDRLDQEHSDKFGRRSSYGGSSYGNSTNNVPASSSTATASTQTQHSGMSPSPSNSYDTSPLPCTTNQNGRENSDRLSTSNGKMSPSRYHVNSMGQRSYSFEASEEDLDVDDKVEELMRRDSSVIKEEIKAFLANRRISQAVVAQVTGISQSRISHWLLQQGSDLSEQKKRAFYRWYQLEKTNPGATLSMRPAPIPIEDPEWRQTPPPVSATPGTFRLRRGSRFTWRKECLAVMESYFNENQYPDEAKREEIANACNAVIQKPEAAILESHGIDVQSPGGHSNSDDVDGNDYSEQDDSTSHSDHQDPISLAVEMAAVNHTILALARQGANEIKTEALDDD

SEQ ID NO: 7 - DNA, 6 bp, artificial
  telomeric repeat
ttaggg

SEQ ID NO: 8 - DNA, 6 bp, artificial
  control sequence
tgtgag

SEQ ID NO: 9 - DNA, 6 bp, artificial
  subtelomeric repeat variant 1
tcaggg

SEQ ID NO: 10 - DNA, 6 bp, artificial
  subtelomeric repeat variant 2
tgaggg

SEQ ID NO: 11 - DNA, 6 bp, artificial
  subtelomeric repeat variant 3
ttgggg

SEQ ID NO: 12 - DNA, 6 bp, Caenorhabditis elegans
ttaggc

SEQ ID NO: 13 - DNA, 46 bp, artificial
  Renilla Luciferase esiRNA forward primer
gctaatacgactcactatagggagaggataactggtccgcagtggt

SEQ ID NO: 14 - DNA, 46 bp, artificial
  Renilla Luciferase esiRNA reverse primer
gctaatacgactcactatagggagacccattcatcccatgattcaa

SEQ ID NO: 15 - DNA, 35 bp, artificial
  esiRNA-1 HOT1 forward primer
tcactatagggagagatttcccaagcagttgttgc

SEQ ID NO: 16 - DNA, 36 bp, artificial
  esiRNA-1 HOT1 reverse primer
tcactatagggagacccagatctgacagctttttgc

SEQ ID NO: 17 - DNA, 35 bp, artificial
  esiRNA-2 HOT1 forward primer
tcactatagggagaggttacctccctgaaagtata

SEQ ID NO: 18 - DNA, 35 bp, artificial
  esiRNA-2 HOT1 reverse primer
tcactatagggagacgttggttattatttactggg

SEQ ID NO: 19 - DNA, 40 bp, artificial
  Homeobox domain deletion forward primer
ctgcgacgagggagtgccaatattgaagcagcaatcctgg

SEQ ID NO: 20 - DNA, 40 bp, artificial
  Homeobox domain deletion reverse primer
tgcttcaatattggcactccctcgtcgcagtcggaaagta

SEQ ID NO: 21 - DNA, 20 bp, artificial
  Terc qPCR primer forward
aagagttgggctctgtcagc

SEQ ID NO: 22 - DNA, 20 bp, artificial
  Terc qPCR primer reverse
gcatgtgtgagccgagtcct

SEQ ID NO: 23 - DNA, 70 bp, artificial
  HOT1 BAC stitching primer forward
cgatttacctggagaaaggagtgcctggctgttatggaaagttacttcaatgagaatcaatacccagatg

SEQ ID NO: 24 - DNA, 70 bp, artificial
  HOT1 BAC stitching primer reverse
tataatacagcatttatgatattctaaagtacttttagagatagaaccaaccctgtgctgctacattgaa

SEQ ID NO: 25 - DNA, 60 bp, artificial
  TTAGGG for primer
ttagggttagggttagggttagggttagggttagggttagggttagggttagggttaggg

SEQ ID NO: 26 - DNA, 60 bp, artificial
  TTAGGG rev primer
aaccctaaccctaaccctaaccctaaccctaaccctaaccctaaccctaaccctaaccct

SEQ ID NO: 27 - DNA, 60 bp, artificial
  GTGAGT for primer
ttgacagtgagtgtgagtgtgagtgtgagtgtgagtgtgagtgtgagtgtgagtgtgagt

SEQ ID NO: 28 - DNA, 60 bp, artificial
  GTGAGT rev primer
aaactcacactcacactcacactcacactcacactcacactcacactcacactcactgtc

SEQ ID NO: 29 - DNA, 60 bp, artificial
  TTAGGC for primer
ttaggcttaggcttaggcttaggcttaggcttaggcttaggcttaggcttaggcttaggc

SEQ ID NO: 30 - DNA, 60 bp, artificial
  TTAGGC rev primer
aagcctaagcctaagcctaagcctaagcctaagcctaagcctaagcctaagcctaagcct

SEQ ID NO: 31 - DNA, 60 bp, artificial
  TTGGGG for primer
ttggggttggggttggggttggggttggggttggggttggggttggggttggggttgggg

SEQ ID NO: 32 - DNA, 60 bp, artificial
  TTGGGG rev primer
aaccccaaccccaaccccaaccccaaccccaaccccaaccccaaccccaaccccaacccc

SEQ ID NO: 33 - DNA, 60 bp, artificial
  TCAGGG for primer
gtcagggtcagggtcagggtcagggtcagggtcagggtcagggtcagggtcagggtcagg

SEQ ID NO: 34 - DNA, 60 bp, artificial
  TCAGGG rev primer
accctgaccctgaccctgaccctgaccctgaccctgaccctgaccctgaccctgaccctg

SEQ ID NO: 35 - DNA, 60 bp, artificial
  TGAGGG for primer
gtgagggtgagggtgagggtgagggtgagggtgagggtgagggtgagggtgagggtgagg

SEQ ID NO: 36 - DNA, 60 bp, artificial
  TGAGGG rev primer
accctcaccctcaccctcaccctcaccctcaccctcaccctcaccctcaccctcaccctc

SEQ ID NO: 37 - protein, 19 aa, artificial [sequence omitted]

SEQ ID NO: 38 - protein, 12 aa, artificial
  peptide fragment of HOT1 (Fig. 2)
HEILHALETLDR

SEQ ID NO: 39 - DNA, 22 bp, artificial
  esiRNA TRF1 forward
tggcaactttaaagaagcagaa

SEQ ID NO: 40 - DNA, 22 bp, artificial
  esiRNA TRF1 reverse
agcttcagtttccatttcaaca

SEQ ID NO: 41 - DNA, 20 bp, artificial
  esiRNA TCAB1 forward
cgaatcgaggagcaagaact

SEQ ID NO: 42 - DNA, 20 bp, artificial
  esiRNA TCAB1 reverse
gggctgaggacatcagagaa

SEQ ID NO: 43 - DNA, 20 bp, artificial
  TCAB1 qPCR primer forward
agccagacacctcctacgtg

SEQ ID NO: 44 - DNA, 20 bp, artificial
  TCAB1 qPCR primer reverse
ggttgaagccacagaagagc

SEQ ID NO: 45 - DNA, 20 bp, artificial
  GAPDH qPCR primer forward
cagcctcaagatcatcagca

SEQ ID NO: 46 - DNA, 20 bp, artificial
  GAPDH qPCR primer reverse
tgtggtcatgagtccttcca

SEQ ID NO: 47 - DNA, 18 bp, artificial
  TS primer
aatccgtcgagcagagtt

SEQ ID NO: 48 - DNA, 30 bp, artificial
  ACX primer
gcgcggcttacccttacccttaccctaacc

(19) United States
(12) Patent Application Publication
(10) Pub. No.: US 2013/0303594 A1
(43) Pub. Date: Nov. 14, 2013

(54) HOT1 AND USES THEREOF

(71) Applicants: Dennis Kappei, Dresden (DE); Frank Buchholz, Dresden (DE); Matthias Mann, Stockdorf (DE); Falk Butter, Munchen (DE)

(72) Inventors: Dennis Kappei, Dresden (DE); Frank Buchholz, Dresden (DE); Matthias Mann, Stockdorf (DE); Falk Butter, Munchen (DE)

(73) Assignee: Max-Planok-Gesellschaft Zur Forderung Der Wissenschaften E.V., Munchen (DE)

(21) Appl. No.: 13/823,431

(22) PCT Filed: Sep. 14, 2011
(86) PCT No.: PCT/EP11/65943

§ 371 Date: Jun. 28, 2013

(30) Foreign Application Priority Data
Sep. 14, 2010 (EP) 10009584.3

(51) Int. Cl. C07K 14/47

(52) U.S. Cl. CPC C07K 14/4703
USPC 514/44 R; 435/7.1

(57) ABSTRACT

The invention relates to polynucleotides and polypeptides as defined in the claims for use in the treatment of a disorder associated with abnormal telomerase activity and/or abnormal telomere length in comparison to healthy subjects in a subject. Moreover, the present invention provides a method of identifying an agent capable of decreasing telomerase activity and/or telomere length, and a method of classifying a cancer as a telomerase-negative cancer, as defined in the claims.

### BACKGROUND OF THE INVENTION

Telomeres are repetitive DNA structures that together with a telomere-specific complex, shelterin, protect the ends of chromosomes. Telomere shortening is mitigated in stem cells and cancer cells through the de novo addition of telomeric repeats by telomerase. Telomere length homeostasis, a crucial process in stem cell biology, aging and cancer, depends on the equilibrium between telomere lengthening (usually due to telomerase activity) and shortening reactions (usually due to replication). Telomerase is usually limiting and, under physiological conditions, acts preferentially on short telomeres due to a well established negative feedback loop mediated in cis by TRF1 and POT1. The latter also interacts with TPP1, which has been shown to be required for recruitment of telomerase to its substrate in vitro and to telomeric chromatin in vivo. However, it is not known that TRF1 actively interacts with telomerase. More importantly, down-regulation of TRF1 in a cell even results in an elongation of the telomeres (van Steensel & de Lange, Nature 385: 740-743). Therefore, TRF1 and HMBOX1 cannot be directly compared in function and knowledge derived from TRF1 cannot be directly transferred to HMBOX1. Thus, despite the increasing number of proteins found to be associated with telomeres, knowledge about the mechanisms involved in telomerase-dependent telomere length homeostasis is still incomplete. In particular, it is not known how the telomerase complex, which assembles and accumulates in nuclear Caja1 bodies (CBs), is recruited to telomeres. Any putative mechanism would need to provide a molecular bridge between the telomerase complex, other components of the Caja1 body, and telomeric structures.

The telomere elongation reaction requires the delivery of the telomerase complex accumulated in Caja1 bodies to telomeres, through an as yet unknown mechanism. Factors promoting this telomerase-telomere interaction are expected to both (i) directly bind telomeric DNA and (ii) physically interact with the telomerase complex found in CBs. HMBOX1 has been previously isolated from a pancreatic cDNA library (Chen et al. Cytogenet Genome Res 114:131-136 (2006)), and its function was postulated as a transcription repressor playing a role in the function of pancreas or pancreatic morphogenesis. Blastp search of the GenBank human protein database revealed that HMBOX1 has the highest sequence similarity to human HNF1β (Hepatocyte Nuclear Factor 1β; 28% identity) and HNF1α (27% identity), respectively. Moreover, orthologs are highly conserved, since sequence similarity of each ortholog to human HMBOX1 (gi:45758726; SEQ ID NO: 2) protein is 99% (Mus musculus; gi:31542626; SEQ ID NO: 3), 81% (Xenopus laevis; gi:50417680; SEQ ID NO: 4), 78% (gallus gallus; gi:50745226; SEQ ID NO: 5), and 77% (Rattus norvegicus; gi:34874315; SEQ ID NO: 6). HMBOX1 is 1,263 by in length, which encodes a putative protein with 420 amino acid residues. The deduced HMBOX1 protein has a calculated molecular mass of 47.25 kDa and contains a homeobox domain between amino acids 267 and 344.

CN 101555486 (A) and CN101555484 (A) describe recombinant human HMBOX1 expression vectors, production of HMBOX1 protein, antibodies directed against human HMBOX1, and the use of HMBOX1 in the regulation and control of natural killer cells. More specifically, CN 101555486 describes how over-expression of HMBOX1 has an effect on the proliferation of natural killer cells and the expression of certain cell surface markers in this context. However, CN 101555486 does not mention HMBOX1 in the context of telomere and telomerase binding, and remains silent with regard to telomeres or cancers with abnormal telomerase activity.

Dejardin & Kingston, Cell 136: 175-186 (2009) could detect ambiguous telomeric association (not to be confused with direct binding) of HMBOX1, and the authors considered association of HMBOX1 with telomeres as being false positive.

Thus, the mechanism of telomere lengthening is not fully understood. The discovery of a factor which is capable of directly binding telomeric DNA and physically interacting with the telomerase complex would open up a complete new approach for the treatment of disorders associated with abnormal telomere lengthening, such as cancer, aging associated diseases or premature aging syndromes, e.g. dyskeratosis congenita.

### SUMMARY OF THE INVENTION

In search for such a factor, the inventors carried out a SILAC-based DNA protein interaction screen and identified HMBOX1, in the priority document referred to as telomeric repeat binding factor 3, or TRF3; and hereafter referred to as Homeobox Telomere binding protein 1, HOT1. HOT1 directly and specifically binds double-stranded telomere repeats. Depletion and overexpression experiments identify HOT1 as a positive regulator of telomere length, i.e. telomerase activity. Furthermore, immuno-precipitation analysis shows that HOT1 physically interacts with components of the active telomerase complex as well as with Caja1 body proteins.

The inventors have identified HOT1 as the first telomere-repeat binding protein that associates both with Caja1 bodies and with telomerase and promotes telomere lengthening. Thus, HOT1 is a putative telomere and/or telomerase recruitment factor that may bridge telomerase to telomeres through binding of both telomeric DNA and the telomerase RNP (FIG. 13). Collectively, these findings suggest that HOT1 mediates telomere-telomerase association in Caja1 bodies.

As noted above, HOT 1 may represent a complete new approach for the treatment of disorders associated with abnormal telomere lengthening and/or telomerase activity, such as cancer, aging associated diseases or premature aging syndromes, e.g. dyskeratosis congenita.

Accordingly, in a first aspect, the invention relates to a polynucleotide encoding a polypeptide comprising an amino acid sequence having at least 70% homology to the amino acid sequence shown in SEQ ID NO: 2, wherein the polypeptide is capable of directly binding to telomere repeats and to the telomerase complex, or a fragment of said polynucleotide,

for use in the treatment of a disorder associated with abnormal telomerase activity and/or abnormal telomere length in comparison to healthy subjects in a subject;
wherein direct binding to telomere repeats is determined by in vitro telomere pulldown, and
wherein direct binding to the telomerase complex is determined by RNA immunoprecipitation and quantitative real-time PCR.

In a second aspect, the invention provides a polypeptide comprising an amino acid sequence having at least 70% homology to the amino acid sequence shown in SEQ ID NO: 2, wherein the polypeptide is capable of directly binding to telomere repeats and to the telomerase complex,

for use in the treatment of a premature-aging syndrome;
wherein direct binding to telomere repeats is determined by in vitro telomere pulldown, and
wherein direct binding to the telomerase complex is determined by RNA immunoprecipitation and quantitative real-time PCR.

In an important third aspect, the invention relates to A polypeptide comprising an amino acid sequence having at least 70% homology to the amino acid sequence shown in SEQ ID NO: 2, wherein the polypeptide is capable of directly binding to telomere repeats and to the telomerase complex,

for use in the treatment of a disorder associated with abnormal telomerase activity and/or abnormal telomere length, wherein the disorder is selected from dyskeratosis congenita, aplastic anemia, and idiopathic pulmonary fibrosis;
wherein direct binding to telomere repeats is determined by in vitro telomere pulldown, and
wherein direct binding to the telomerase complex is determined by RNA immunoprecipitation and quantitative real-time PCR.

Further, the invention relates to a method of identifying an agent capable of decreasing telomerase activity, comprising the steps of
(i) contacting a candidate agent with a polypeptide comprising an amino acid sequence having at least 70% homology to the amino acid sequence shown in SEQ ID NO: 2, wherein the polypeptide is capable of directly binding to telomere repeats and to the telomerase complex;
   wherein direct binding to telomere repeats is determined by in vitro telomere pulldown, and/or
   wherein direct binding to the telomerase complex is determined by RNA immunoprecipitation and quantitative real-time PCR; and
(ii) determining if the polypeptide subjected to step (i) is capable of
   (a) binding to telomere repeats, and/or
   (b) binding to the telomerase complex;
      wherein a reduced binding determined in step (ii) (a) and/or step (ii) (b) compared to that of a corresponding polypeptide not subjected to step (i) is indicative of an agent capable of decreasing the telomerase activity.

In a final aspect, the invention also relates to a method of classifying a cancer as a telomerase-negative cancer, comprising the step of

determining the co-localisation of promyelocytic leukaemia (PML) bodies and a polypeptide comprising an amino acid sequence having at least 70% homology to the amino acid sequence shown in SEQ ID NO: 2, wherein the polypeptide is capable of directly binding to telomere repeats and to the telomerase complex,
wherein direct binding to telomere repeats is determined by in vitro telomere pulldown, and
wherein direct binding to the telomerase complex is determined by RNA immunoprecipitation and quantitative real-time PCR;
in a cell comprised in a specimen of the cancer to be classified;
wherein a co-localisation is indicative of a telomerase-negative cancer.

### DETAILED DESCRIPTION OF THE PREFERRED EMBODIMENTS

In a first aspect, the invention relates to a polynucleotide encoding a polypeptide comprising an amino acid sequence having at least 70% homology to the amino acid sequence shown in SEQ ID NO: 2, wherein the polypeptide is capable of directly binding to telomere repeats and to the telomerase complex, or a fragment of said polynucleotide, for use in the treatment of a disorder associated with abnormal telomerase activity and/or abnormal telomere length in comparison to healthy subjects in a subject;

wherein direct binding to telomere repeats is determined by in vitro telomere pulldown, and
wherein direct binding to the telomerase complex is determined by RNA immunoprecipitation and quantitative real-time PCR.

The term “polynucleotide” as used herein refers to any polynucleotide capable of encoding the polypeptide of the invention, for example DNA, RNA, cDNA, cRNA, DNA/RNA hybrids, or the like. The polynucleotide may be double stranded, or single stranded, such as the (+) or the (−) strand of the polynucleotide. Moreover, the polynucleotide may be a genomic sequence, which may still comprise introns, or a non-genomic polynucleotide, which does not comprise any introns. A polynucleotide, as meant herein has a length starting from 15 nucleotides up to more than about 1300 nucleotides, such as more than 1500 nucleotides, or more than 2000 nucleotides, such as more than 2500 nucleotides, more than 3000 nucleotides, more than 4000 nucleotides, more than 5000 nucleotides, more than 6000 nucleotides, more than 7000 nucleotides, up to more than 10.000 nucleotides. The polynucleotide may be modified at the base moiety, sugar moiety, or phosphate backbone. The polynucleotide may include other appending groups such as peptides, or agents facilitating transport across the cell membrane, hybridization-triggered cleavage agents, or intercalating agents.

The term “polypeptide” as used herein, refers to a single linear chain of amino acids, linked by a peptide bond. As used herein, a polypeptide comprises at least 300 amino acids, preferably at least 350 amino acids, more preferably at least 400 amino acids, such as 420 amino acids, or at least 450 amino acids, such as at least 500 amino acids, at least 600 amino acids, at least 700 amino acids, at least 800 amino acids, at least 900 amino acids, at least 1000 amino acids, or even up to 1500 amino acids or up to 2000 amino acids. C-terminal or N-terminal extensions, such as tags, or fusions with another polypeptide, such as a fluorescent polypeptide, are also contemplated.

Further, the polypeptide comprises an amino acid sequence having at least 70% homology, preferably at least 75% homology, such as at least 80% homology, more preferably at least 85% homology, such as at least 90% homology, even more preferably at least 95% homology, and most preferably at least 98% homology to the amino acid sequence shown in SEQ ID NO: 2. Most preferably, the polypeptide comprises an amino acid sequence which is identical to SEQ ID NO: 2.

Generally, amino acid sequence has “at least x % homology” with another amino acid sequence, when the sequence identity between those to aligned sequences is at least x %, based on the full length of SEQ ID NO: 2. Such an alignment can be performed using for example publicly available computer homology programs such as the “BLAST” program provided at the NCBI homepage at http://www.ncbi.nlm.nih.gov/blast/blast.cgi, using the default settings provided therein. Further methods of calculating sequence homology percentages of sets of amino acid sequences are known in the art.

In order to maintain biological activity, the polypeptide may preferably comprise conservative substitutions or semi-conservative substitutions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Typical semi-conservative and conservative substitutions are:

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Furthermore, the skilled person will appreciate that glycines at sterically demanding positions should not be substituted and that proline should not be introduced into parts of the protein which have an alpha-helical or a beta-sheet structure. In another preferred embodiment, the polypeptide consists of the amino acid sequence shown in SEQ ID NO: 2. In addition, the polypeptide is capable of directly binding to telomere repeats. Direct binding to telomere repeats may be determined by in vitro telomere pulldown, as described in the example section.

Briefly, chemically synthesized telomere oligonucleotides (Metabion) are annealed and polymerized by T4 ligase (Fermentas) and biotinylated with biotin-dATP (Invitrogen) by Klenow fragment (Fermentas) following the manufacturer instructions. 25 μg baits are immobilized on 750 μg paramagnetic streptavidin beads (Dynabeads MyOne C1, Invitrogen) and subsequently incubated with 400 μg of SILAC-labeled nuclear extract in PBB buffer (150 mM NaCl, 50 mM Tris/HCl pH 8.0, 10 mM MgCl₂, 0.5 percent NP-40, Complete Protease Inhibitor-EDTA [Roche]) for 2 hours at 4° C. on a rotation wheel. After three times washing with PBB buffer bead fractions are pooled, bound proteins boiled at 80° C. in 1× LDS buffer (Invitrogen) and separated on a 4%-12% gradient gel (Novex, Invitrogen). In-gel digestion and MS analysis is performed essentially as previously described by Butter et al., EMBO Rep. 11, 305-311 (2010). Peptides are desalted on stage tips and analyzed by nanoflow liquid chromatography on an EASY-nLC system from Proxeon Biosystems coupled to a LTQ-Orbitrap XL or a Orbitrap-Velos (Thermo Electron). Peptides are separated on a C18-reversed phase column (15 cm long, 75 μm inner diameter, packed with ReproSil-Pur C18-AQ 3 μm resin directly mounted on the electrospray ion source. A 107 min gradient from 2% to 60% acetonitrile in 0.5% acetic acid is used at a flow of 200 nl/min. The LTQ-Orbitrap XL is operated with a TopS MS/MS spectra acquisition method in the linear ion trap per MS full scan in the orbitrap while for the Orbitrap-Velos a Top10 acquisition method with HCD fragmentation is used. The raw files are processed with MaxQuant and searched with the Mascot search engine (Matrix Science) against IPI human v3.37 protein database concatenated with a decoy of the reversed sequences.

The choice of telomere repeat in the assay will depend on the origin of the polypeptide. That is, if the polypeptide originated from a vertebrate, the telomeric repeat to be used in the assay is the telomeric repeat of said vertebrate. In general, the telomere repeat of vertebrates is TTAGGG (SEQ ID NO: 7).

Additionally, the polypeptide is capable of directly binding to the telomerase complex. The direct binding to the telomerase complex may be determined by RNA immunoprecipitation and quantitative real-time PCR, as described in the examples section.

Briefly, immunoprecipitations are carried out using the Dynabeads Protein G immunoprecipitation kit (Invitrogen). Beads are treated with 10 μg rabbit anti-HOT1 (MPI-CBG Antibody Facility), rabbit anti-DKC1 (ab64667, abcam) or rabbit IgG (sc-66931, Santa Cruz) in NET-2 buffer (150 mM NaCl, 50 mM Tris/HCl pH 7.5, 0.05 percent NP-40, Complete Protease Inhibitor-EDTA [Roche]) and subsequently incubated with NET-2 extracted and sonicated lysates for 3 hours at 4° C. on a rotation wheel, followed by five washes with NET-2 buffer. RNA is extracted by a standard phenol/chloroform procedure. cDNA synthesis is carried out using the SuperScript III first strand synthesis kit (Invitrogen) using a Terc specific primer (for primer sequences see Table 2 below). At the end of cDNA synthesis samples are treated with RNase Cocktail (Applied Biosystems). For qPCR, primers are used at 70 nM concentration together with the Absolute qPCR SYBR green mix (Abgene) on a Mx3000p real-time PCR system (Stratagene). Target gene mRNA levels are normalized against quantification of GAPDH mRNA levels for housekeeping. However, the skilled person will be aware of further methods well-known in the art for determining direct binding to the telomerase complex. For example, immunoprecipitation of telomerase activity may be measured by the telomerase repeat amplification protocol (TRAP), or analogous methods.

The term “telomerase complex” is well known in the art. As illustrated in FIG. 13, the telomerase complex may include for example DKC1, NHP2, NOP10, GAR1, TERT, and/or TERC.

It is well known that abnormal telomerase activity and/or abnormal telomere length gives rise to a number of disorders. Whereas increased telomerase activity and/or telomere length in a cell of a subject suffering from an disorder associated with abnormal telomerase activity and/or abnormal telomere length compared to a corresponding cell in a healthy subject may lead to immortality of the cell (i.e. the cell is a cancer cell), decreased telomerase activity and/or telomere length may result in a premature-aging syndrome. Accordingly, in one preferred embodiment, the disorder associated with abnormal telomerase activity and/or abnormal telomere length is a premature-aging syndrome, more preferably selected from the group consisting of dyskeratosis congenita, Werner syndrome, Cockayne syndrome, Bloom syndrome, ataxia telangiectasia, ataxia telangiectasia-like disorder, Nijmegen breakage syndrome, Cri du chat syndrome, Fanconi anemia, xeroderma pigmentosum, Hoyeraal-Hreidarsson syndrome, Li-Fraumeni syndrome, Niedernhofer syndrome, and Hutchinson-Gilford progeria syndrome.

Thus, in case of a premature-aging syndrome, it is desirable to increase the telomerase activity and/or telomere length. As shown in the examples, HOT1 is supposed to be one of the factors limiting telomerase activity and/or telomere lengthening. Further, it is demonstrated by the inventors that over-expression of HOT1 results in an increased telomerase activity, as detectable in an increase of telomere length. Therefore, in case of a premature-aging syndrome, said polynucleotide is preferably used in order to increase the expression of said polypeptide in the cells of said subject.

In another preferred embodiment, the disorder associated with abnormal telomerase activity and/or abnormal telomere length is dyskeratosis congenita, aplastic anemia, or idiopathic pulmonary fibrosis. In particular, aplastic anemia and idiopathic pulmonary fibrosis are quite similar disorders as compared to dyskeratosis congenita.

In another preferred embodiment, the disorder associated with abnormal telomerase activity and/or abnormal telomere length is cancer, preferably selected from the group of cancers consisting of lung cancer, breast cancer, colorectal cancer, pancreatic cancer, ovarian cancer, prostate cancer, bladder cancer, uterine cancer, liver cancer, kidney cancer, endometrial cancer, thyroid cancer, oral or pharyngeal cancer, esopharyngeal cancer, brain cancer, skin melanoma, leukaemia, fibrosarcoma, and non-Hodgkin lymphoma.

In case of a cancer, telomerase activity and/or telomere length are increased in the cancer cell compared to a corresponding cell in a healthy subject. Accordingly, it is desired to decrease or even abrogate the telomerase activity, and/or to shorten telomere length in such a cancer cell. Therefore, in a preferred embodiment, the polynucleotide or fragment thereof is used to reduce the expression of said polypeptide in the cancer cells of said subject. One approach is to employ antisense polynucleotides or oligonucleotides (in case of a fragment of the polynucleotide) derived from the polynucleotide of the invention in order to inhibit expression of the polypeptide of the invention. Methods to select, test and optimize putative antisense sequences starting from SEQ ID NO: 1 or SEQ ID NO: 2 are routine. Nucleic acid constructs may be used to express an antisense molecule of interest, or the antisense polynucleotide or fragment thereof as such may be administered to a cell. Multiple antisense constructs or polynucleotides may be employed together. The antisense sequences may range from about 6 to about 50 nucleotides, and may be as large as 100 or 200 nucleotides, or larger. They may correspond to full-length coding sequences and/or may be genomic sequences that comprise non-coding sequences.

Another approach involves double stranded RNAs called small interfering RNAs. A siRNA is a double-stranded RNA molecule comprising self-complementary sense and antisense regions, wherein the antisense region comprises nucleotide sequence that is complementary to nucleotide sequence in a target nucleic acid molecule or a portion thereof, and the sense region has a nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary. The siRNA can be assembled from a single polynucleotide, where the self-complementary sense and antisense regions of the siRNA are linked by means of a nucleic acid based or non-nucleic acid-based linker. The siRNA may be a polynucleotide having a hairpin secondary structure, i.e. having self-complementary sense and antisense regions. The siRNA may be a circular single-stranded polynucleotide having two or more loop structures and a stem comprising self-complementary sense and antisense regions, wherein the circular polynucleotide can be processed either in vivo or in vitro to generate an active siRNA molecule capable of mediating RNAi. In certain embodiments, the siRNA molecule comprises separate sense and antisense sequences or regions, wherein the sense and antisense regions are covalently linked by nucleotide or non-nucleotide linker molecules as is known in the art, or are alternatively non-covalently linked by ionic interactions, hydrogen bonding, van der Waals interactions, hydrophobic interactions, and/or stacking interactions. RNAi molecules may be used to inhibit gene expression, using conventional procedures.

In order to introduce an antisense molecule, or siRNAs into a cancer cell of interest, conventional methods of gene transfer may be used, which may include virus-mediated gene transfer, for example, by using retroviruses, lentiviruses, recombinant adenovirus, and adeno-associated virus (AAV) vectors. In addition to virus-mediated gene transfer, physical means well-known in the art can be used for direct gene transfer, including administration of plasmid DNA and particle-bombardment mediated gene transfer. Furthermore, electroporation or calcium phosphate transfection, both well-known means to transfer genes into a cell in vitro, may be used. Further, gene transfer may alternatively be achieved by using “carrier mediated gene transfer”. Preferred carriers are targeted liposomes such as immunoliposomes, or polycations such as asiaglycoprotein/polylysine. Liposomes have been used to encapsulate and deliver a variety of materials to cells, including nucleic acids and viral particles. Preformed liposomes that contain synthetic cationic lipids form stable complexes with polyanionic DNA. Cationic liposomes, liposomes comprising some cationic lipid, that contained a membrane fusion-promoting lipid dioctadecyldimethyl-ammonium-bromide (DDAB) have efficiently transferred heterologous genes into eukaryotic cells and can mediate high level cellular expression of transgenes, or mRNA, by delivering them into a variety of cultured cell lines.

In a preferred embodiment the fragment of the polynucleotide of the invention is an esiRNA. esiRNAs are enzymatically prepared siRNAs, resulting from digestion of long double stranded RNAs with, e.g., an RNaseIII family enzyme in vitro. The polynucleotide fragment may range in size from about 6 to about 50 nucleotides, such as from about 10 to about 60 nucleotides, from about 15 to about 70 nucleotides, from about 15 to about 80 nucleotides, from about 20 to about 90 nucleotides, or about 25 to about 95 nucleotides, and may be as large as 100 or 200 nucleotides, or larger. The detailed protocol of esiRNA production has been previously published (Kittler, R., et al. Nat. Methods 2, 779-784 (2005)). Briefly, optimal region for designing the esiRNA may be chosen using the Deqor design algorithm (Henschel, A., et al. Nucleic Acids Res. 32, W113-120 (2004)) in order to fulfill two criteria: to obtain the most efficient silencing trigger in terms of silencing efficiency, and to get lowest chances to cross-silence other genes. The most favourable fragment are used to design the gene specific primers by the Primer3 algorithm (http://frodo.wi.mit.edu/cgi-bin/primer3/primer3_www.cgi). As shown in the examples, two esiRNAs for HOT1 were designed and synthesized (for primer sequences see Table 2 below). PCR products for the esiRNA production may be sequenced using an Applied Biosystems 3730 Genetic Analyzer (Applied Biosystems) according to the manufacturer's instructions.

The subject to be treated may be any subject suffering or being prone to suffer from a disorder associated with abnormal telomerase activity and/or abnormal telomere length. However, preferably the subject is a vertebrate, e.g. chicken, more preferably the subject is a mammal, such as a horse, cow, pig, mouse, rat, guinea pig, cat, dog, goat, sheep, or a primate. In a most preferred embodiment, the subject is a human.

In a second aspect, the invention relates to the polypeptide itself as further defined in the first aspect. Accordingly, the invention provides a polypeptide comprising an amino acid sequence having at least 70% homology to the amino acid sequence shown in SEQ ID NO: 2, wherein the polypeptide is capable of directly binding to telomere repeats and to the telomerase complex,

for use in the treatment of a premature-aging syndrome;
wherein direct binding to telomere repeats is determined by in vitro telomere pulldown, and
wherein direct binding to the telomerase complex is determined by RNA immunoprecipitation and quantitative real-time PCR.

Since the expression level of the polypeptide of the invention positively correlates with telomere length in a cell, the skilled person will acknowledge that the polypeptide itself cannot be used to decrease the level of the polypeptide in the invention in order to reduce the telomere length in a cell. Accordingly, the polypeptide of the invention may be used in the treatment of disorders associated with abnormal reduced telomerase activity and/or telomere length in comparison to healthy subjects in a subject, e.g., in the treatment of a premature-aging syndrome in a subject.

In a preferred embodiment, the premature-aging syndrome is selected from the group consisting of dyskeratosis congenita, Werner syndrome, Cockayne syndrome, Bloom syndrome, ataxia telangiectasia, ataxia telangiectasia-like disorder, Nijmegen breakage syndrome, Cri du chat syndrome, Fanconi anemia, xeroderma pigmentosum, Hoyeraal-Hreidarsson syndrome, Li-Fraumeni syndrome, Niedernhofer syndrome, and Hutchinson-Gilford progeria syndrome.

Likewise, a polypeptide comprising an amino acid sequence having at least 70% homology to the amino acid sequence shown in SEQ ID NO: 2, wherein the polypeptide is capable of directly binding to telomere repeats and to the telomerase complex, may also be used in the treatment of a disorder associated with abnormal telomerase activity and/or abnormal telomere length, wherein the disorder is selected from dyskeratosis congenita, aplastic anemia, and idiopathic pulmonary fibrosis; wherein direct binding to telomere repeats is determined by in vitro telomere pulldown, and wherein direct binding to the telomerase complex is determined by RNA immunoprecipitation and quantitative real-time PCR.

The terms “polypeptide”, “x % homology”, “capable of directly binding to telomere repeats”, “capable of directly binding to the telomerase complex”, and “subject” have already been explained and defined above.

In one preferred embodiment, the polypeptide comprises an amino acid sequence having at least 70% homology, preferably at least 75% homology, such as at least 80% homology, more preferably at least 85% homology, such as at least 90% homology, even more preferably at least 95% homology, and most preferably at least 98% homology to the amino acid sequence shown in SEQ ID NO: 2. Most preferably, the polypeptide comprises an amino acid sequence which is identical to SEQ ID NO: 2. In still another most preferred embodiment, the polypeptide consists of the amino acid sequence shown in SEQ ID NO: 2. The polynucleotide or polypeptide may be formulated in a pharmaceutical composition. Such a pharmaceutical composition may comprise at least one additional carrier, excipient or diluent. The choice of carrier may depend upon route of administration and concentration of the active agent(s) and the pharmaceutical composition may be in the form of a lyophilised composition or an aqueous solution. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

The composition may also contain more than one active compound. For example, the composition may additionally comprise a telomerase inhibitor, or a telomerase activator.

For example, Geron Corporation is currently conducting two clinical trials involving telomerase inhibitors. One uses a vaccine (GRNVAC1) and the other uses a lipidated drug (GRN163L). An example of a telomerase activator is TA-65, a telomerase activator agent derived from the Chinese astragalus plant.

As noted above, HOT1's unique property is its direct binding to both the telomeric repeats as well as to the telomerase complex. Furthermore, it could be demonstrated that the expression of HOT1, i.e. its biological activity, is directly linked with the telomere length in a telomerase positive cell. Accordingly, it is to be expected that an agent capable of blocking or affecting the binding to either one or even both of these binding partners will result in a reduction in telomerase activity and/or telomere length.

Thus, the invention also relates to a method of identifying an agent capable of decreasing telomerase activity and/or telomere length, comprising the steps of
(i) contacting a candidate agent with a polypeptide comprising an amino acid sequence having at least 70% homology to the amino acid sequence shown in SEQ ID NO: 2, wherein the polypeptide is capable of directly binding to telomere repeats and to the telomerase complex;
   wherein direct binding to telomere repeats is determined by in vitro telomere pulldown, and/or
   wherein direct binding to the telomerase complex is determined by RNA immunoprecipitation and quantitative real-time PCR;
(ii) determining if the polypeptide subjected to step (i) is capable of
   (a) binding to telomere repeats, and/or
   (b) binding to the telomerase complex;
      wherein a reduced binding determined in step (ii) (a) and/or step (ii) (b) compared to that of a corresponding polypeptide not subjected to step (i) is indicative of an agent capable of decreasing the telomerase activity and/or telomere length.

In a preferred embodiment, the polypeptide is as defined in the first and second aspect. Binding to telomere repeats and binding to the telomerase complex may be determined as described above and in the examples section.

The term “decreasing telomerase activity and/or telomere length” is intended to mean any significant detectable reduction in telomerase activity and/or telomere length in a cell, preferably in a telomerase-positive cell. Further, a decrease of telomerase activity and/or a decrease in telomere length may be monitored conducting a telomeric quantitative FISH assay, as described in the examples in the passage headed “Telomeric quantitative FISH”.

The candidate agent may be a small molecule, either isolated from natural sources or developed synthetically, e.g., by combinatorial chemistry. The skilled person in the field of drug discovery and development will understand that the precise source of candidate agents is not critical to the methods of the invention. Accordingly, virtually any number of chemical extracts or compounds can be used in the method described herein. Examples of such candidate agents include, but are not limited to, plant-, fungal-, prokaryotic- or animal-based extracts, fermentation broths, and synthetic compounds, as well as modifications of existing compounds. Also encompassed by the term “candidate agent” are saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, e.g., Biotics (Sussex, UK), Xenova (Slough, UK), Harbor Branch Oceangraphics Institute (Ft. Pierce, Fla.), and PharmaMar, U.S.A. (Cambridge, Mass.). Alternatively, natural and synthetically produced libraries may be generated, according to methods well known in the art.

A subset of about 10% of cancer cells is not maintaining their telomeres by telomerase reactivation but uses instead the recombination based ALT (alternative lengthening of telomeres) mechanism. In these cells telomeres associate with PML (promyelocytic leukemia) bodies, which are therefore also called ALT-associated PML bodies (APBs) in this particular context. For the detection of an ALT-positive cell one can colocalize telomeres or the previously known telomere-binding proteins TRF1 and TRF2 with APBs. However, only a subset of telomeres within one cell is associated with APBs at a given time, reducing the robustness of this readout. However HOT1 associates with APBs in ALT-positive cells exclusively in a standard immunofluorescence assay, making HOT1 a good candidate for a strong diagnostic marker of ALT cancer cells. The result of such an diagnostic assay may provide the physician with useful information for the further treatment of the patient. For example, administration of a telomerase inhibitor is considered being ineffective in telomerase-negative cancer.

Accordingly, in a final aspect, the invention provides a method of classifying a cancer as a telomerase-negative cancer, comprising the step of

determining the co-localisation of promyelocytic leukaemia (PML) bodies and a polypeptide comprising an amino acid sequence having at least 70% homology to the amino acid sequence shown in SEQ ID NO: 2, wherein the polypeptide is capable of directly binding to telomere repeats and to the telomerase complex,
wherein direct binding to telomere repeats is determined by in vitro telomere pulldown, and
wherein direct binding to the telomerase complex is determined by RNA immunoprecipitation and quantitative real-time PCR; in a cell comprised in a specimen of the cancer to be classified;
wherein a co-localisation is indicative of a telomerase-negative cancer.

The term “telomerase-negative cancer”, as used herein, is intended to mean a cancer,

wherein the cells of said cancer maintain their telomeres by a recombination based ALT (alternative lengthening of telomeres) mechanism. Said term is not to be construed as said cells do not express any telomerase at all. Rather, it is intended to mean that the ALT mechanism is the predominant mechanism, by which the telomeres of the cell are maintained.

Preferably, the polypeptide is as defined in the first or second aspect. PML-bodies are well known to the skilled person. PML bodies and their function in ALT cells is also reviewed, for example, in Draskovic, et al, PNAS 106(37): 15726-15731 (2009).

The specimen may be a biopsy tissue sample, such as a fine needle aspiration, core needle biopsy, vacuum assisted biopsy, direct and frontal biopsy, or a surgical biopsy, e.g. a specimen derived from a primary tumor tissue which has been removed by surgery. Alternatively, the specimen may be a skin biopsy, such as a shave biopsy, punch biopsy, incisional biopsy, excisional biopsy, curettage biopsy, or scoop excision.

The cancer may be selected from the group of cancers consisting of lung cancer, breast cancer, colorectal cancer, pancreatic cancer, ovarian cancer, prostate cancer, bladder cancer, uterine cancer, liver cancer, kidney cancer, endometrial cancer, thyroid cancer, oral or pharyngeal cancer, esopharyngeal cancer, brain cancer, skin melanoma, leukaemia, fibrosarcoma, and non-Hodgkin lymphoma.

In a preferred embodiment, the co-localisation is determined by immunofluorescence, as also described in example 3. Antibodies directed against the polypeptide of the invention may be produced as described, for example, in the example section, in the paragraph headed “Antibody production”.

In the following, the present invention is illustrated by figures and examples which are not intended to limit the scope of the present invention.

### DESCRIPTION OF THE FIGURES

FIG. 1: A schematic of the quantitative-SILAC-based DNA interaction screen with DNA oligonucleotides containing either the telomeric repeat or a control sequence. Specific interaction partners are differentiated from background binders by having a SILAC ratio other than 1:1.

FIG. 2: MS spectra of representative peptides validated by MS/MS from the ‘forward’ pull-down experiment. The heavy peptide partners are detected (red dots) while the light partner is barely observable (blue dots).

FIG. 3: Logarithmic two-dimensional plot for ratios of individual proteins obtained in the ‘forward’ (telomeric sequence incubated with heavy-labeled extract and control sequence with light-labeled extract) and the ‘reverse’ (vice versa) pull-down. Known shelterin components cluster together with HOT1 demonstrating enrichment at the telomere sequence in comparison to the control. TIN2 and POT1 had infinite ratios in the reverse experiment and TPP1 in the forward experiment (indicated by arrows).

FIG. 4: Sequence specific pull-down of recombinant HOT1. Proteins were incubated with telomeric repeats (TTAGGG; SEQ ID NO: 7), the control sequence (TGTGAG; SEQ ID NO: 8), the subtelomeric repeat variants TCAGGG (SEQ ID NO: 9), TGAGGG (SEQ ID NO: 10) and TTGGGG (SEQ ID NO: 11) as well as the C. elegans telomere repeat TTAGGC (SEQ ID NO: 12).

FIG. 5: Quantification of HOT1 esiRNA knock-down effiency using a HOT1-LAP cell line. (left: Western blot; right: quantification).

FIG. 6: Quantification of telomere length by quantitative telomeric FISH after transient knockdown of HOT1. The distributions of fluorescence intensities, in arbitrary units of fluorescence (a.u.f.), of individual telomeres from a total of 30 metaphases per treatment are displayed; the average intensity is indicated in red. For the gene-specific knockdowns, changes of average telomere signal intensity relative to the RLuc (Renilla Luciferase) control are shown (left). Representative FISH images are shown for each treatment and signal free ends are indicated by arrows (right). Examples of individual chromosomes are magnified and the respective chromosomes are marked by rectangles (right). Scale bars represent 5 μm.

FIG. 7: Summary of the quantification of signal free ends per metaphase after gene-specific knockdown.

FIG. 8: Results of telomere length measurements after transient overexpression of Flag-HOT1 and Flag-HOT 1 ΔHomeobox.

FIG. 9: Sequence specific pull-down of Flag-HOT1 and Flag-HOT1ΔHomeobox. Proteins were incubated with either telomeric repeats (TTAGGG; SEQ ID NO: 7) or a control oligonucleotide (TGTGAG; SEQ ID NO: 8).

FIG. 10: Summary of SILAC-based protein-protein interactions. Identification and normalized SILAC ratios are indicated for HOT1 (bait) and the identified interaction partner relevant for telomere biology.

FIG. 11: Validation of the mass spectrometry identifications by conventional immunoprecipitation. Nuclear HeLa extracts were subject to immunoprecipitation with either a HOT1 or an IgG antibody and immunoblotted for DKC1 and Ku70.

FIG. 12: Quantification of TERC enrichment after RNA immunoprecipitation (RIP) using antibodies against HOT1 and DKC1 and the enrichments were normalized against RIP using an IgG control. A representative result is shown.

FIG. 13: Model summarizing the relationship of HOT1 to its interaction partners and its novel role as a telomere binding protein that acts as a positive regulator of telomere length.

FIG. 14: Twodimensional interaction plot of SILAC HOTHP. The antibody against HOT1 immunoprecipitated HOT1 (colored red) along with Dyskerin complex (black), the Ku70-Ku80 heterodimer (blue) and the core Caja1 body protein Coilin (brown).

FIG. 15: Caja1 body proteins are enriched in the HOT1-IP. (a) Twodimensional interaction plots for 4 complexes that are annotated CORUM and enriched in the HOT1-IP, putatively part of Caja1 bodies. (b) Comparison of the previously purified complexes (CORUM) demonstrates strong overlap between Ribosome/NOP45p associated pre-rRNA complex and C complex spliceosome/Spliceosome. (c) Coverage of the annotated complexes by the HOT1-IP.

FIG. 16: Chromatin immunoprecipitation (ChIP) of telomeric DNA using both a rabbit and a mouse anti-HOT1 antibody and anti-H4K₂₀me3 (positive control). The sonication efficiency of chromatin samples from HeLa cells was verified by an agarose gel. Representative slot blot images for ChIP with both the rabbit and mouse anti-HOT1 antibodies are shown. Input dilutions demonstrate the linearity of the signal detections. Fold enrichments of telomeric DNA were calculated in comparison to the species-specific IgG controls. Error bars represent the standard deviation of biological replicates (rabbit antibodies: n=8; mouse antibodies: n=2). All enrichments are statistically significant with p<0.05 (student's t-test).

FIG. 17: Co-localization analysis of telomeres and HOT1 in HeLa cells by immunoFlSH staining. A representative image illustrating the co-localization between several HOT1 foci (green) and telomeres (red) is shown. DAPI (blue) is used as nuclear counter stain. Co-localization events are indicated by arrows. Scale bars represent 1 μm. The quantification of the frequency of co-localization events was done after 3D reconstruction of the acquired z-stacks (n=147). The average value is indicated by a red bar.

FIG. 18: Quantification of telomerase activity enrichment in immunoprecipitation using antibodies against DKC1 and HOT1. Enrichments are normalized to immunoprecipitations using an IgG control. Error bars represent the standard deviation of three independent experiments. All enrichments are statistically significant with p<0.05 (student's t-test).

### EXAMPLES

### Cell Culture

HeLa cells (Epitheloid carcinoma, cervix) were cultivated in 4.5 g/l glucose Dulbecco's Modified Eagle's Medium (D-MEM) supplemented with 10% fetal bovine serum, 100 U/ml Penicillin and 100 μg/ml Streptomycin (Gibco) at 37° C. and 5% CO₂. For SILAC labelling HeLa S3 cells were incubated in RPMI 1640 (-Arg, -Lys) medium containing 10% dialyzed fetal bovine serum (Gibco) supplemented with 84 mg/1 ¹³C₆¹⁵N₄ L-arginine and 50 mg/l ¹³C₆¹⁵N₂ L-lysine (Sigma Isotec or Euriso-top) or the corresponding non-labeled amino acids, respectively. RUE murine embryonic stem cells were grown in DMEM (-Arg, -Lys) medium containing 10% dialyzed FBS (Gibco) supplemented with 40 mg/1 ¹³C₆¹⁵N₄ L-arginine and 80 mg/l ¹³C₆¹⁵N₂ L-lysine (Sigma Isotec or Euris-top) or the corresponding non-labeled amino acids, respectively. Cells were harvested and nuclear extracts were prepared as described (Butter, F., et al. EMBO Rep. 11, 305-311 (2010)).

### Telomere Pulldown

Chemically synthesized oligonucleotides (Metabion) were annealed and polymerized by T4 ligase (Fermentas) and biotinylated with biotin-dATP (Invitrogen) by Klenow fragment (Fermentas) following the manufacturer instructions. 25 μg baits were immobilized on 750 μg paramagnetic streptavidin beads (Dynabeads MyOne C1, Invitrogen) and subsequently incubated with 400 μg of SILAC-labeled nuclear extract in PBB buffer (150 mM NaCl, 50 mM Tris/HCl pH 8.0, 10 mM MgCl₂, 0.5 percent NP-40, Complete Protease Inhibitor-EDTA [Roche]) for 2 hours at 4° C. on a rotation wheel. After three times washing with PBB buffer bead fractions were pooled, bound proteins boiled at 80° C. in 1×LDS buffer (Invitrogen) and separated on a 4%-12% gradient gel (Novex, Invitrogen).

### MS Data Acquisition

In-gel digestion and MS analysis was performed essentially as previously described by Butter et al., supra. Peptides were desalted on stage tips and analyzed by nanoflow liquid chromatography on an EASY-nLC system from Proxeon Biosystems coupled to a LTQ-Orbitrap XL or a Orbitrap-Velos (Thermo Electron). Peptides were separated on a C18-reversed phase column (15 cm long, 75 μm inner diameter, packed in-house with ReproSil-Pur C18-AQ 3 μm resin (Dr. Maisch) directly mounted on the electrospray ion source. We used a 107 min gradient from 2% to 60% acetonitrile in 0.5% acetic acid at a flow of 200 nl/min. The LTQ-Orbitrap XL was operated with a Top5 MS/MS spectra acquisition method in the linear ion trap per MS full scan in the orbitrap while for the Orbitrap-Velos a Top10 acquisition method with HCD fragmentation was used.

### MS Spectrum and Data Analysis

The raw files were processed with MaxQuant and searched with the Mascot search engine (Matrix Science) against IPI human v3.37 protein database concatenated with a decoy of the reversed sequences. Carbamidomethylation was set as fixed modification while methionine oxidation and protein N-acetylation were considered as variable modifications. The search was performed with an initial mass tolerance of 7 ppm mass accuracy for the precursor ion and 0.5 Da for the MS/MS spectra obtained with CID fragmentation and 20 ppm for the MS/MS spectra in the HCD fragmentation mode. Search results were processed with MaxQuant filtered with a false discovery rate of 0.01. Prior to statistical analysis, known contaminants and reverse hits were removed. The protein ratios of a ‘forward’ experiment and the ‘reverse’ experiment were plotted in R (prerelease version 2.8.0). Only proteins identified with at least 2 unique peptides and 2 quantitation events were plotted for the telomere IP (quality filter).

### Recombinant Protein Expression and Deletion Variant Construction

The HOT1 clone was obtained from the ORFeome collection (IOH40784, Invitrogen). The sequence was subcloned into SLIC-compatible pETM44 vector via SLIC cloning (Li, M. Z. & Elledge, S. J. Nat. Methods 4, 251-256 (2007)) and expressed in E. coli Rosetta at 18° C. E. coli extracts with overexpressed recombinant proteins were used for binding studies on variant DNA motives.

The HOT1 ORFeome clone was LR recombined into a gateway compatible pcDNA3.1 vector with N-terminal flag tag (gift of Christian Brandts). A flag-tagged TRF 3 homeobox deletion variant was constructed by PCR amplification of the pcDNA3.1-flag vector using primers with site-specific overhangs (for primer sequences see Table 2 below) following the QuickChange II Site-directed mutagenesis kit protocol (Stratagene). The constructs were sequence verified using an Applied Biosystems 3730 Genetic Analyzer (Applied Biosystems) according to the manufacturer's instructions.

### Antibody Production

His-MBP-tagged HOT1 (petM44 vector construct) was expressed in E. coli Rosetta at 18° C. in a fermentation tank. Cell pellet was lysed with Avestin and the soluble fraction subjected to affinity purification using Ni-sepharose. The elution fraction was concentrated with an Amicon Ultra 15 concentrator column and dialyzed into buffer containing 50 mM K₂PO₃, 20 mM NaCl, 10 percent glycerine, 1 mM TCEP and protein inhibitors. Purified MBP-HOT1 was injected into rabbits for immunization and the rabbits were ultimately sacrificed. The Antibody was affinity purified using His-MBP-HOT1 and MBP immobilized on HiTRAP desalting columns (GE Healthcare). First the serum was applied to the His-MBP-HOT1 column, eluted and applied to the MBP column. The flow-trough was quantified and used for subsequent experiments.

Purified MBP-HOT1 was injected into mice for immunization and the mice were ultimately sacrificed. Immortalized hybrid cells (hybridomas) were obtained by the fusion of B-cells from the spleen of an immunized mouse with a myeloma cell line, that itself does not produce antibodies, using PEG and AH selection (Sigma Aldrich). Hybridoma clones were generated and screened using the Meso Scale Discovery platform (Meso Scale Diagnostics) by comparing affinity to MBP-HIS-HOT1 and MBP-HIS-Katanin as an unspecific negative control. Positive clones were subcloned by limiting dilution and retested using the MSD platform. Based on the subcloned hybridoma cell lines antibodies were purified using HiTRAP protein G columns (GE Healthcare) followed by acid elution.

esiRNA Synthesis

The detailed protocol of esiRNA production has been previously published (Kittler, R., et al. Nat. Methods 2, 779-784 (2005)). Briefly, optimal region for designing the esiRNA were chosen using the Deqor design algorithm (Henschel, A., et al. Nucleic Acids Res. 32, W113-120 (2004)) in order to fulfill two criteria: to obtain the most efficient silencing trigger in terms of silencing efficiency, and to get lowest chances to cross-silence other genes. The most favourable fragment were used to design the gene specific primers by the Primer3 algorithm (http://frodo.wi.mit.edu/cgi-bin/primer3/primer3_www.cgi). Two esiRNAs for HOT 1 were designed and synthesized (for primer sequences see Table 2 below). PCR products for the esiRNA production were sequenced using an Applied Biosystems 3730 Genetic Analyzer (Applied Biosystems) according to the manufacturer's instructions. All positions of sequence trace files were confirmed by manual inspection.

esiRNA and Plasmid Transfection

For all transfections 40,000 HeLa cells were seeded in 3.5 cm² dish and incubated over night before transfection. For esiRNA transfection 20 μl Oligofectamine (Invitrogen) were diluted in 100 μl OptiMEM (Invitrogen) and incubated for 5 min at room temperature. In a separate tube 1.5 μg esiRNA were diluted in 100 μl OptiMEM. Solutions were combined, mixed and incubated for 20 min at room temperature after which the transfection mix was evenly distributed over the dish.

The Flag-HOT1 and the homeobox deletion variant construct were transfected using either Effectene (Qiagen) or Lipofectamine 2000 (Invitrogen) as the transfection reagent according to the manufacturers' instructions.

### Telomeric Quantitative FISH

For metaphase preparation cells were incubated for 4 h with 200 nM nocodazole in order to induce mitotic arrest. A hypotonic shock was achieved in 0.03 M sodium citrate at 37° C. for 40 min. Cells were fixed in an ethanol/acetic acid solution (3:1) and washed three times in this fixing reagent. Metaphase spreads were obtained by dropping suspensions of fixed cells onto clean glass slides.

The Q-FISH procedure was carried out as described, using a Alexa488-O-β-(CCCTAA)3 PNA probe (Panagene) (Londono-Vallejo, J. A., et al. Nucleic Acids Res 29, 3164-3171 (2001)).

Telomeric signals were quantified using the iVision software (Chromaphor). Telomere signals were segmented manually and average pixel intensities from every segment were quantified. For each metaphase the average background intensity was determined and subtracted from individual telomere signals. Statistical analyses were done using the Wilcoxon rank-sum test.

The number of signal free ends per metaphase was determined by manual inspection of the same metaphase images that were used for telomere signal intensity quantification.

### Protein Immunoprecipation

Immunoprecipitations were carried out using the Dynabeads Protein G immunoprecipitation kit (Invitrogen). 50 μl of beads were treated with 10 μg rabbit anti-HOT1 (MPI-CBG Antibody Facility) or rabbit IgG (sc-66931, Santa Cruz; 2729s, Biolabs) in PBB buffer (150 mM NaCl, 50 mM Tris/HCl pH 8.0, 10 mM MgCl₂, 0.5 percent NP-40, Complete Protease Inhibitor-EDTA [Roche]) and subsequently incubated with 400 μg HeLa nuclear extract (SILAC labeled if followed by MS analysis) for 2 hours at 4° C. on a rotation wheel, followed by three washes with PBB buffer. For MS bead fractions were pooled, bound proteins were eluted and separated on a 4%-12% gradient gel (Novex, Invitrogen). For co-IP experiments followed by Western blot bound proteins were simply eluted and subjected to Western blot analysis.

### Western Blot and Antibodies

For Western Blot samples were boiled in Laemmli buffer and subjected to SDS-Page (NuPage 4-12% Bis-Tris gels; Invitrogen). Gels were blotted to nitrocellulose (Protran; Schleicher & Schuell), blocked in 5% non-fat milk in PBST for 1 h at RT and incubated overnight at 4° C. in primary antibody. The following primary antibodies were used: mouse anti-GFP (Roche Diagnostics, 1:4,000 dilution), mouse anti-DMlalpha tubulin (MPI-CBG Antibody Facility, 1:50,000 dilution), mouse anti-Ku70 (sc17789, Santa Cruz, 1:1,000 dilution) and mouse anti-Flag (M2, Sigma-Aldrich, 1:2,000). The next day membranes were washed 3 times for 10 min each in 5% milk PBST and incubated for 1 h at RT with secondary antibody (goat anti-mouse antibody conjugated to horseradish peroxidase, Biorad, 1:4,000). Membranes were washed three times for 10 min each in PBST followed by one PBS wash. Bands were visualised with enhanced chemiluminescence Western Blotting Detection Reagents (GE Healthcare). As a molecular weight standard Spectra Multicolor Broad Range Protein Ladder (Fermentas) was used. For the quantification of HOT1 knockdown efficiency band intensities of western blot images were quantified with ImageJ 1.33u (National Institute of Health).

### Chromatin ImmunoPrecipitation (ChIP)

Cells were crosslinked with 1% formaldehyde (Thermo Scientific) for 10 min at 37° C. and the reaction was stopped by adding glycine to a final concentration of 0.125 M for 5 min at room temperature. Nuclei were prepared from fixed and washed cells by homogenization in cell lysis buffer (5 mM PIPES pH 8, 85 mM KCl, 0.5% NP-40) and centrifugation at 1800 g for 10 min. Finally, nuclei were lyzed in 900 μl nuclei lysis buffer (50 mM Tris-HCl pH 8, 10 mM EDTA pH 8, 1% SDS) and lysates were sonicated for 15 min (30 sec on/30 sec off) in a Diagenode water bath-sonicator at speed 5. Following a centrifugation at 14000 rpm for 10 min, the cleared supernatants were snap-frozen in liquid nitrogen and stored at −80° C. Sonication efficiency was routinely monitored by DNA gel-electrophoresis to ensure that the bulk of DNA fragments was between 100 and 500 bp. Sonicated chromatin containing 30-50 μg DNA was diluted 10 times in ChIP dilution buffer (16.7 mM Tris-HCl pH 8, 16.7 mM NaCl, 1.2 mM EDTA pH 8, 1.1% Triton X-100, 0.01% SDS) and pre-cleared for 1 hour, rotating at 4° C., with 50 μl blocked beads (Protein A agarose 50% slurry [Millipore] incubated for 2 hours with 5 mg/ml BSA) before the overnight incubation with 5 μg of antibody. The following antibodies were used: rabbit anti-HOT1, mouse anti-HOT1 (both MPI-CBG Antibody Facility), rabbit anti-Histone H4K20me3 (ab9053, abcam), rabbit IgG (ab37415, abcam) or mouse IgG (ChromPure, Jackson ImmunoResearch) The bound material was recovered after a 2 hours incubation, rotating at 4° C., with 100 μl blocked beads. The beads were washed, for 10 minutes in each of the following Wash buffers: Low Salt Buffer (20 mM Tris-HCl pH 8, 150 mM NaCl, 2 mM EDTA pH 8, 1% Triton X-100, 0.1% SDS), High Salt Buffer (20 mM Tris-HCl pH 8, 500 mM NaCl, 2 mM EDTA pH 8, 1% Triton X-100, 0.1% SDS), LiCl Buffer (10 mM Tris-HCl pH 8, 0.25 M LiCl, 1 mM EDTA pH 8, 1% NP-40, 1% Na Deoxycholate) and twice, 5 min each, in TE. ChIPed material was eluted by two 15 min incubations at room temperature with 250 μl elution buffer (0.1 M NaHCO₃, 1% SDS). Chromatin was reverse-crosslinked by adding 20 μl of 5M NaCl and incubated at 65° C. for at least 4 hours and DNA was submitted to RNase and proteinase K digestion and extracted by phenol-chloroform. Purified DNA recovered by ChIP was denatured in 0.2 M NaOH by heating to 100° C. for 10 min and spotted onto a positively charged Biodyne B nylon membrane (Pierce). Membranes were hybridized at 42° C. in 6×SSC, 0.01% SDS with 20 pmol of DIG-labeled telomeric Crich LNA probe (36 bp, Exiqon). Following hybridization washes (twice 5 min in 2×SSC, 0.01% SDS and once 2 min in 0.1×SSC, 0.01% SDS) the signal was revealed using the anti-DIG-AP antibodies (Roche) and CDP-Star (Roche) following the manufacturer's instructions. Images were obtained using the Luminescent image analyzer LAS-4000 mini (GE Healthcare). Band intensities were quantified with ImageJ 1.33u (National Institutes of Health). Statistical analyses were done using student's t-test.

### Immunofluorescence and ImmunoFISH Stainings

For immunofluorescence stainings cells were seeded 24 hours before the treatment on either glass coverslips (0.17 mm, assorted glass, Thermo Scientific) or in Lab-Tek II chambered coverglass chambers (Labtek). After a brief wash with 1×PBS cells were fixed in 10% formalin solution (Sigma Aldrich) for 10 min at room temperature, followed by two washes with 1×PBS+30 mM glycine. Cells were then permeabilized with 1×PBS+0.5% Triton X-100 for 5 min at 4° C., followed by again two washes with 1×PBS+30 mM glycine. Afterwards cells were blocked in blocking solution (1×PBS, 0.2% fish skin gelatine [Sigma Aldrich]) for 15 min at room temperature. Primary antibodies were diluted in blocking solution and incubated for 1 hour at room temperature. The following primary antibodies were used: mouse anti-HOT1 (MPI-CBG Antibody Facility, 1:2000) and rabbit anti-Coilin (sc-32860, Santa Cruz, 1:500). Cells were washed 3 times for 3 min each in blocking solution followed by a 30 min incubation at room temperature with secondary antibodies, which were diluted in blocking solution. As secondary antibodies fluorescent labeled donkey anti rabbit-IgG or donkey anti mouse-IgG antibodies with either Alexa488 or Alexa594 as fluorochoromes (Invitrogen) were used. After 3 final washes for 3 min each in blocking solution slides were briefly rinsed in destilled water and mounted using DAPI Prolong Gold Antifade Reagent (Invitrogen). In the case of Labtek chambers samples were incubated for 5 min with blocking solution containing 1 μg/ml DAPI, followed by one wash in blocking solution.

If FISH stainings were combined with immunofluorescence stainings (immunoFlSH), the FISH labeling was carried out directly after the IF protocol. Cells were washed once in PBS and then hydrated with consecutive washes in 70%, 90% and 100% ethanol for 5 min each followed by a standard telomeric FISH protocol as previously described (Londono-Vallejo, J. A., et al. Nucleic Acids Res 29, 3164-3171 (2001).

All images were acquired with a DeltaVision Core Microscope (Applied Precision, Olympus IX71 microscope) using a 100×/1.4 UPlanSApo oil immersion objective. Z-stacks (0.2 μm optical sections) were collected and deconvolved using softWoRx (Applied Precision). Zstacks were reconstructed in 3D using Imarisi (Bitplane) and co-localization events were determined for signals above the background using the colocalization function.

### RNA Immunoprecipation and Quantitative Real-Time PCR

Immunoprecipitations were carried out using the Dynabeads Protein G immunoprecipitation kit (Invitrogen). Beads were treated with 10 μg rabbit anti-HOT1 (MPI-CBG Antibody Facility), rabbit anti-DKC1 (ab64667, abcam) or rabbit IgG (sc-66931, Santa Cruz) in NET-2 buffer (150 mM NaCl, 50 mM Tris/HCl pH 7.5, 0.05 percent NP-40, Complete Protease Inhibitor-EDTA [Roche]) and subsequently incubated with NET-2 extracted and sonicated lysates for 3 hours at 4° C. on a rotation wheel, followed by five washes with NET-2 buffer. RNA was extracted by a standard phenol/chloroform procedure. cDNA synthesis was carried out using the SuperScript III first strand synthesis kit (Invitrogen) using a Terc specific primer (for primer sequences see Table 2 below). At the end of cDNA synthesis samples were treated with RNase Cocktail (Applied Biosystems). For qPCR primers were used at 70 nM concentration together with the Absolute qPCR SYBR green mix (Abgene) on a Mx3000p real-time PCR system (Stratagene). Target gene mRNA levels were normalized against quantification of GAPDH mRNA levels for housekeeping.

### Immunopreciptiation of Telomerase Activity and Quantitative TRAP Assay

For the immunoprecipitation of telomerase activity HeLa cells were lysed in lysis buffer (50 mM Tris-HCl (pH 8.0), 150 mM NaCl and 1% NP-40 supplemented with Complete Protease Inhibitor-EDTA [Roche]) for 30 min on ice followed by a 30 min centrifugation step in a table-top centrifuge at 4° C. and 20,000 g. Per IP 50 μl of sepharose protein A beads (Millipore) were used. Beads were washed 3 times with PBS prior to use and incubated with 5 mg/ml BSA (in PBS) for 1 hour at 4° C. on a rotating wheel, while lysates were pre-cleared with uncoated beads for 1 hour at 4° C. on a rotating wheel. Per IP 1 mg lysate was incubated with 10 μg rabbit IgG (sc-66931, Santa Cruz), rabbit anti-DKC1 (sc-48794, Santa Cruz) or rabbit anti-HOT1 (MPI-CBG Antibody Facility) in PBS for 2 hours at 4° C. on a rotating wheel. BSA-coated beads were added, followed by a second incubation for 2 hours at 4° C. on a rotating wheel. Beads were then washed once with PBS, twice with lysis buffer and again once with PBS and finally recovered in 150 μl Chaps buffer (Chemicon). The quantitative TRAP (Telomerase Repeat Amplification Protocol) assay was carried out using GoTaq qPCR Master Mix (Promega) and both the TS and ACX primer at 200 nM. The reaction was run on a Mx3000p real-time PCR system (Stratagene) with the following protocol: 25° C. for 20 min, 95° C. for 10 min and 32 cycles with 95° C. for 30 sec, 60° C. for 30 sec and 72° C. for 1 min. Statistical analyses were done using student's t-test.

### BAC TransgeneOmics

The following BACs (bacterial artificial chromosomes) were used in this study: For HOT1 RP11-789B24 (Invitrogen) and for DKC1 RP11-107C18 (BACPAC Resource Center). A LAP (localization and affinity purification) cassette was inserted as a C-terminal fusion using recombineering. Isolated BAC DNA was transfected and selected for stable integration as described (Poser, I. et al. Nat. Methods 5, 409-415). The BAC RP11-789B24 does not cover the entire HOT1 gene and was complemented by insertion of a cDNA fragment covering the missing coding and 3′UTR sequence (for primer sequences see Table 2 below).

TABLE 2
Oligonucleotides used in this study

| Target | forward primer (5′-->3′) | reverse primer (5′-->3′) |
|---|---|---|
| Renilla Luciferase | GCTAATACGACTCACTATAGG | GCTAATACGACTCACTATAGG |
| esiRNA | GAGAGGATAACTGGTCCGCAG | GAGACCCATTCATCCCATGAT |
| | TGGT (SEQ ID NO: 13) | TCAA (SEQ ID NO: 14) |
| esiRNA-1 HOT1 | TCACTATAGGGAGAGATTTCC | TCACTATAGGGAGACCCAGAT |
| | CAAGCAGTTGTTGC (SEQ | CTGACAGCTTTTTGC (SEQ |
| | ID NO. 15) | ID NO: 16) |
| esiRNA-2 HOT1 | TCACTATAGGGAGAGGTTACC | TCACTATAGGGAGACGTTGGT |
| | TCCCTGAAAGTATA (SEQ | TATTATTTACTGGG (SEQ |
| | ID NO: 17) | ID NO: 18) |
| Homeobox | CTGCGACGAGGGAGTGCCAAT | TGCTTCAATATTGGCACTCCC |
| domain deletion | ATTGAAGCAGCAATCCTGG | TCGTCGCAGTCGGAAAGTA |
| | (SEQ ID NO: 19) | (SEQ ID NO: 20) |
| Terc qPCR | AAGAGTTGGGCTCTGTCAGC | GCATGTGTGAGCCGAGTCCT |
| primer | (SEQ ID NO: 21) | (SEQ ID NO: 22) |
| HOT1 BAC | CGATTTACCTGGAGAAAGGAG | TATAATACAGCATTTATGATA |
| stitching primers | TGCCTGGCTGTTATGGAAAGT | TTCTAAAGTACTTTTAGAGAT |
| to add missing | TACTTCAATGAGAATCAATAC | AGAACCAACCCTGTGCTGCTA |
| CDS and 3′UTR | CCAGATG (SEQ ID NO: | CATTGAA (SEQ ID NO: |
| | 23) | 24) |
| esiRNA TRF1 | TGGCAACTTTAAAGAAGCAGA | AGCTTCAGTTTCCATTTCAAC |
| | A (SEQ ID NO: 37) | A (SEQ ID NO: 38) |
| esiRNA TCAB1 | CGAATCGAGGAGCAAGAACT | GGGCTGAGGACATCAGAGAA |
| | (SEQ ID NO: 39) | (SEQ ID NO: 40) |
| TCAB1 qPCR | AGCCAGACACCTCCTACGTG | GGTTGAAGCCACAGAAGAGC |
| primer | (SEQ ID NO: 41) | (SEQ ID NO: 42) |
| GAPDH qPCR | CAGCCTCAAGATCATCAGCA | TGTGGTCATGAGTCCTTCCA |
| primer | (SEQ ID NO: 43) | (SEQ ID NO: 44) |

| Sequence motif | primer sequence (5′-->3′) |
|---|---|
| TTAGGG for | TTAGGGTTAGGGTTAGGGTTAGGGTTAGGGTTAGGGTTAGGGTTAGGG |
| | TTAGGGTTAGGG (SEQ ID NO: 25) |
| TTAGGG rev | AACCCTAACCCTAACCCTAACCCTAACCCTAACCCTAACCCTAACCCT |
| | AACCCTAACCCT (SEQ ID NO: 26) |
| GTGAGT for | TTGACAGTGAGTGTGAGTGTGAGTGTGAGTGTGAGTGTGAGTGTGAGT |
| | GTGAGTGTGAGT (SEQ ID NO: 27) |
| GTGAGT rev | AAACTCACACTCACACTCACACTCACACTCACACTCACACTCACACTC |
| | ACACTCACTGTC (SEQ ID NO: 28) |
| TTAGGC for | TTAGGCTTAGGCTTAGGCTTAGGCTTAGGCTTAGGCTTAGGCTTAGGC |
| | TTAGGCTTAGGC (SEQ ID NO: 29) |
| TTAGGC rev | AAGCCTAAGCCTAAGCCTAAGCCTAAGCCTAAGCCTAAGCCTAAGCCT |
| | AAGCCTAAGCCT (SEQ ID NO: 30) |
| TTGGGG for | TTGGGGTTGGGGTTGGGGTTGGGGTTGGGGTTGGGGTTGGGGTTGGGG |
| | TTGGGGTTGGGG (SEQ ID NO: 31) |
| TTGGGG rev | AACCCCAACCCCAACCCCAACCCCAACCCCAACCCCAACCCCAACCCC |
| | ACCCTCACCCTC (SEQ ID NO: 32) |
| TCAGGG for | GTCAGGGTCAGGGTCAGGGTCAGGGTCAGGGTCAGGGTCAGGGTCAGG |
| | GTCAGGGTCAGG (SEQ ID NO: 33) |
| TCAGGG rev | ACCCTGACCCTGACCCTGACCCTGACCCTGACCCTGACCCTGACCCTG |
| | ACCCTGACCCTG (SEQ ID NO: 34) |
| TGAGGG for | GTGAGGGTGAGGGTGAGGGTGAGGGTGAGGGTGAGGGTGAGGGTGAGG |
| | GTGAGGGTGAGG (SEQ ID NO: 35) |
| TGAGGG rev | ACCCTCACCCTCACCCTCACCCTCACCCTCACCCTCACCCTCACCCTC |
| | ACCCTCACCCTC (SEQ ID NO: 36) |
| TS primer | AATCCGTCGAGCAGAGTT (SEQ ID NO: 45) |
| ACX primer | GCGCGGCTTACCCTTACCCTTACCCTAACC (SEQ ID NO: 46) |

### Example 1

### Identification of HMBOX1 (HOT 1) as a Direct and Specific Telomere Repeat Binding Protein

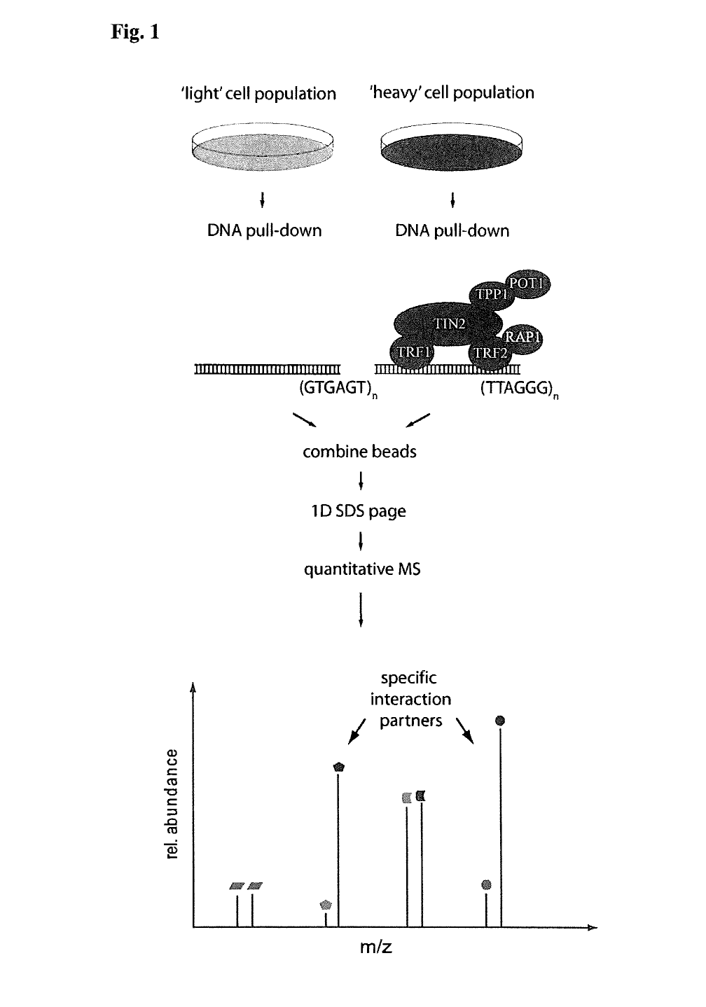

To discover novel DNA-binding proteins we previously used SILAC-based quantitative mass spectrometry (Ong, S. E. et al. Mol. Cell. Proteomics 1, 376-386 (2002)) to identify factors binding to particular functional DNA fragments (Butter, F., et al. EMBO Rep. 11, 305-311 (2010); Mittler, G., et al. Genome Res. 19, 284-293 (2009)). In this assay, specific binding of proteins is detected by incubation of heavy amino acid encoded cell lysates with the bait sequence, using as a control a light amino acid encoded cell lysate. Specific binders display a differential isotope ratio whereas background binders have a one-to-one ratio. In the present attempt to identify telomere binding proteins the inventors used polymerized biotinylated double-stranded oligonucleotides of the telomeric sequence (TTAGGG; SEQ ID NO: 7) in comparison to a scrambled control sequence (TGTGAG; SEQ ID NO: 8). Both oligonucleotides were immobilized on paramagnetic streptavidin beads and incubated with SILAC-labeled human nuclear extracts from HeLa cells. After mild washing, bead fractions were combined and captured proteins analyzed by quantitative, high-resolution mass spectrometry (Butter, F., et al., supra) (FIG. 1). The inventors identified all six core shelterin components with a high SILAC ratio (greater than 10) in the ‘forward’ as well as in the ‘reverse’ experiment, in which we had switched the labels (data not shown).

TABLE 1
Telomere-binding proteins from HeLa and ES cells.
Summary of the mass spectrometry data from the SILAC-based
DNA-protein interaction screens carried out with nuclear HeLa
and ES cell extracts. The number of identified unique peptides,
quantification events and normalized SILAC ratios are displayed
each for the ‘forward’ (telomeric sequence incubated with
heavy-labeled extract and control sequence with light-labeled
extract) and the ‘reverse’ (vice versa) pull-down.

| | Forward experiment | | | Reverse experiment | | |
|---|---|---|---|---|---|---|
| | Unique | | | Unique | quan- | |
| | peptides | quantifi- | SILAC | peptides | tifi- | SILAC |
| Protein | identified | cations | ratio | identified | cations | ratio |

HeLa S3 cells

| | | | | | | |
|---|---|---|---|---|---|---|
| HOT1 | 8 | 10 | 22.2 | 10 | 6 | 0.02 |
| TRF1 | 15 | 21 | 11.6 | 14 | 7 | 0.02 |
| TRF2 | 9 | 55 | 17.7 | 10 | 29 | 0.02 |
| RAP1 | 19 | 26 | 15.2 | 19 | 17 | 0.02 |
| TIN2 | 3 | 4 | 7.3 | 3 | 3 | (L)* |
| TPP1 | 2 | 2 | (H)* | 3 | 3 | 0.08 |
| POT1 | 4 | 4 | 10.3 | 10 | 10 | (L)* |

Mouse ES cells

| | | | | | | |
|---|---|---|---|---|---|---|
| HOT1 | 14 | 22 | 20.5 | 14 | 20 | 0.11 |
| TRF1 | 21 | 83 | 28.4 | 23 | 102 | 0.02 |
| TRF2 | 30 | 47 | 21.6 | 26 | 51 | 0.08 |
| RAP1 | 20 | 23 | 13.9 | 20 | 20 | 0.04 |
| TIN2 | 1 | 27 | 18.9 | 1 | 26 | 0.04 |
| TPP1 | 12 | 15 | 17.0 | 14 | 15 | 0.01 |
| POT1a | 23 | 31 | 18.6 | 24 | 45 | 0.05 |
| POT1b | 11 | 8 | 5.8 | 13 | 13 | 0.10 |

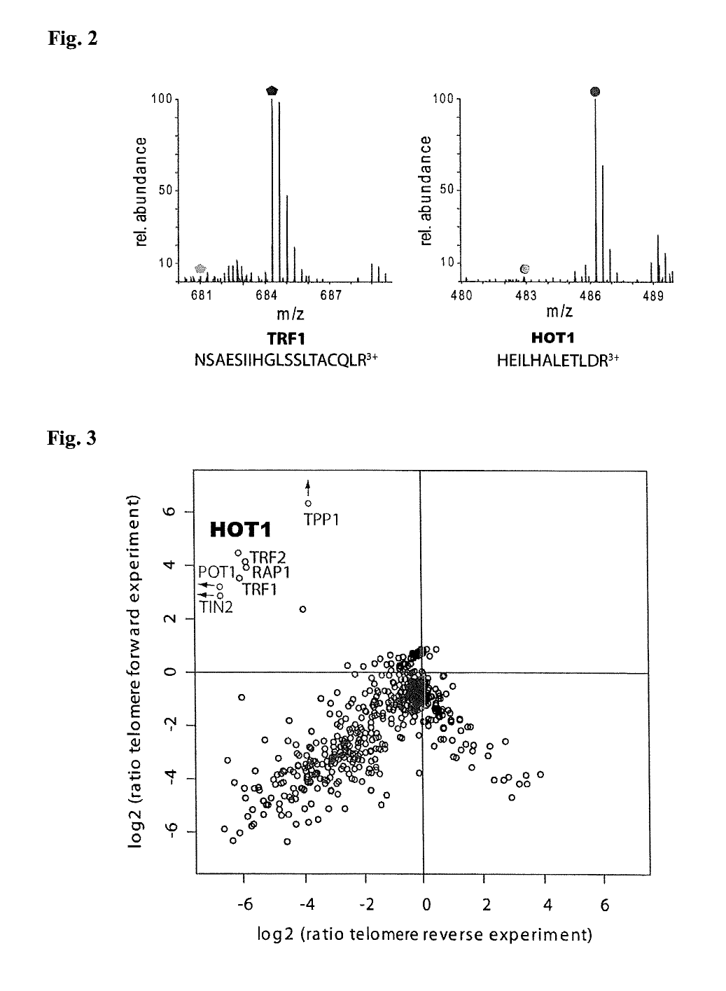

In contrast, none of the proteins known to interact with shelterin were identified with SILAC ratios sufficiently high to be consistent with telomere binding, demonstrating that this approach is very stringent and exclusively detected telomere-repeat binding proteins and their strong interaction partners (FIG. 3). In addition to the shelterin components, we found the protein HMBOX1 with a high SILAC ratio that clustered with those of the shelterin components (Table 1 above, FIG. 2, FIG. 3), indicating that this protein must either strongly associate with the shelterin complex or bind specifically to TTAGGG (SEQ ID NO: 7) repeats.

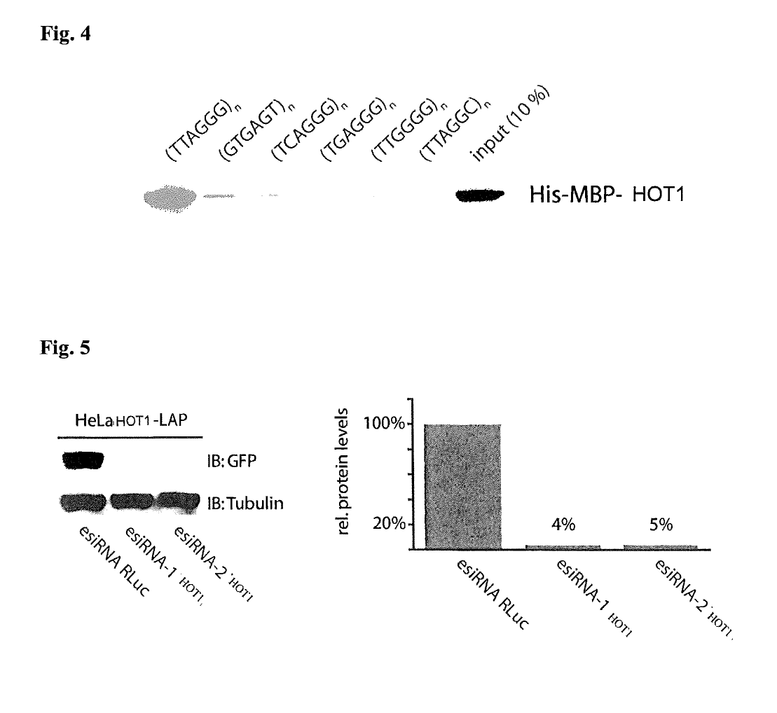

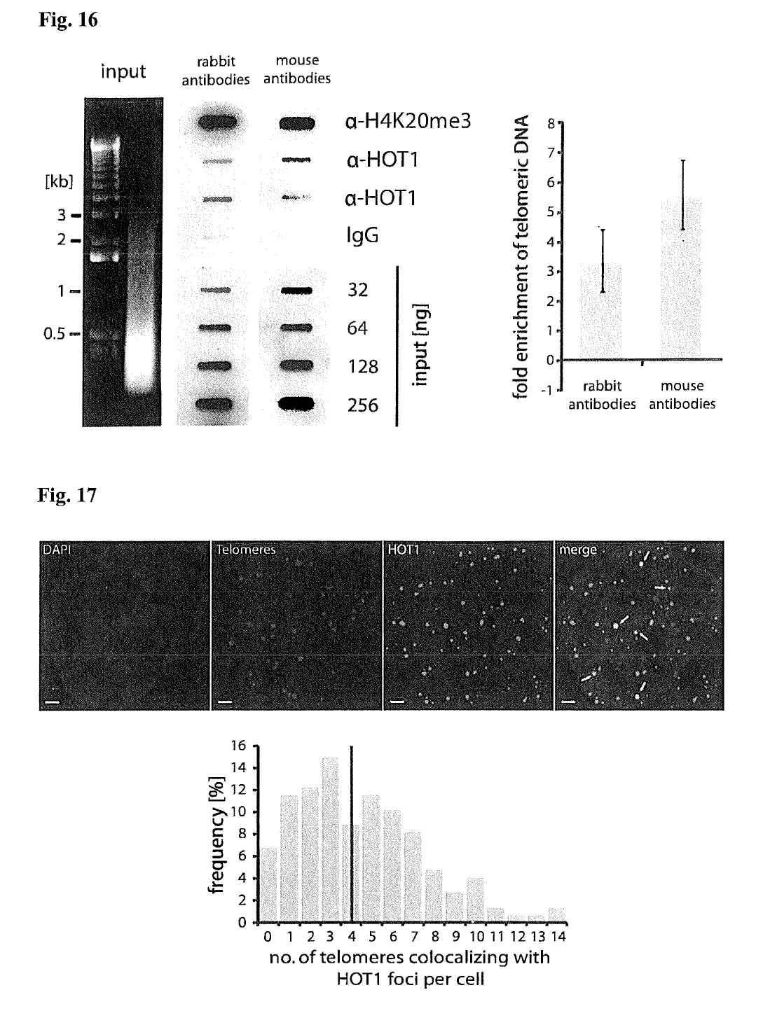

HMBOX1 contains a homeobox domain (Chen et al., supra), suggesting that it may directly bind DNA. To determine whether HMBOX1 was detected in our assay because of a strong association with the shelterin complex or direct binding to TTAGGG (SEQ ID NO: 7) repeats, we performed DNA binding assays with HMBOX1 in vitro. Recombinant HMBOX1 bound specifically to telomeric repeats, whereas no binding was detected for the negative control repeat fragments (TGTGAG; SEQ ID NO: 8). In addition, no binding to the subtelomeric variant repeats TCAGGG (SEQ ID NO: 9), TGAGGG (SEQ ID NO: 10) and TTGGGG (SEQ ID NO: 12) nor to the C. elegans telomere TTAGGC (SEQ ID NO: 13) repeat sequence was detected (FIG. 4). Thus, HMBOX1 is a direct and specific telomere repeat binding protein. We therefore refer to this protein as Homeobox Telomere binding protein 1 (HOT1). To test whether HOT1 associates with telomeres in vivo, the inventors performed chromatin immunoprecipitation (ChIP) experiments with HeLA cells using two independent antibodies directed against HOT1. We detected on average a 3.4-fold and 5.6-fold enrichment of telomeric DNA for a polyclonal rabbit and a monoclonal mouse antibody, respectively. (FIG. 16).

To further substantiate the interaction between HOT1 and telomeres in vivo we analyzed HOT1 intracellular localization by immunoFlSH (immunohistochemistry combined with Fluorescence In Situ Hybridization) microscopy. HOT1 frequently associated with a subset of telomeres, as we observed in about 90% of all cells co-localization of HOT1 with telomeric DNA. On average we detected 4.5 HOT1 and telomeric foci co-localizing per cell. (FIG. 17). Together these data indicate that HOT1 is a direct telomere-binding protein in vitro and in vivo.

To verify that HOT1 interaction was not cancer-, cell- or species-specific, the inventors repeated the telomere-binding assay with nuclear extracts derived from mouse embryonic stem cells. As observed with HeLa cell extracts, all six components of the shelterin complex and HOT1 were identified with SILAC ratios indicating specific binding to the telomere repeats (Table 1, above). In the mouse embryonic stem cells we also identified the two paralogues POT1a and POT1b, which resulted from a gene duplication of the POT1 gene in the rodent lineage, underscoring the specificity of our assay for direct telomere binding proteins. Thus, HOT1 is a telomere repeat binding protein conserved in mammalian cells.

### Example 2

### Functional Characterisation of HOT1

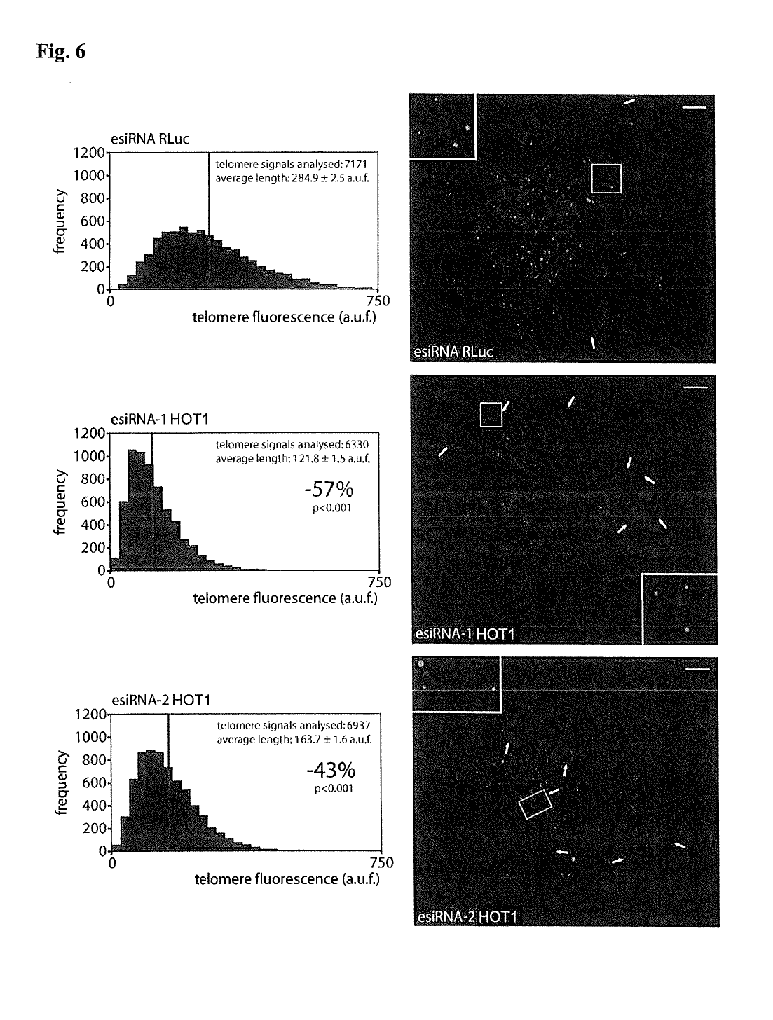

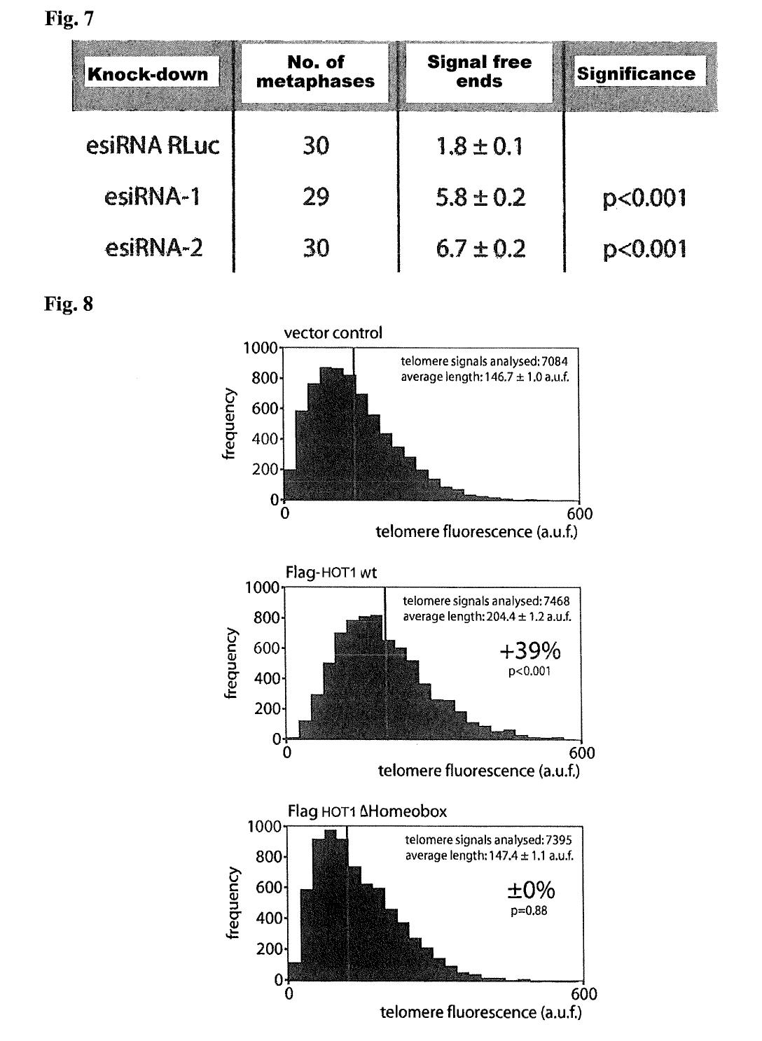

Next, we tested a potential function of HOT1 in telomere homeostasis, by depleting the protein in HeLa cells with endoribonuclease-prepared siRNA (esiRNA) (Kittler, R., et al. Nat. Methods 2, 779-784 (2005)) (FIG. 5). Three days after transfection, metaphase spreads were prepared for telomere length measurements by quantitative telomere specific Fluorescence In Situ Hybridization (FISH) (Londono-Vallejo, J. A., et al. Nucleic Acids Res 29, 3164-3171 (2001)). Knockdown of HOT1 resulted in significant telomere shortening (FIG. 6) with FISH signals reduced on average by 58% and 43% by two independent esiRNAs, respectively. Consistent with this finding we also observed a significant increase in the appearance of signal-free chromosome ends upon HOT1 knockdown (FIG. 6, FIG. 7). In a complementary experiment the inventors transiently overexpressed Flag-HOT1 in HeLa cells and analyzed telomere length three days after transfection. Coherent with shortening upon HOT1 depletion, telomeres were elongated upon its overexpression, which led to an increase in telomeric FISH signals by 39% on average (FIG. 8). Collectively, these findings demonstrate that HOT1 acts as a positive regulator of telomere length.

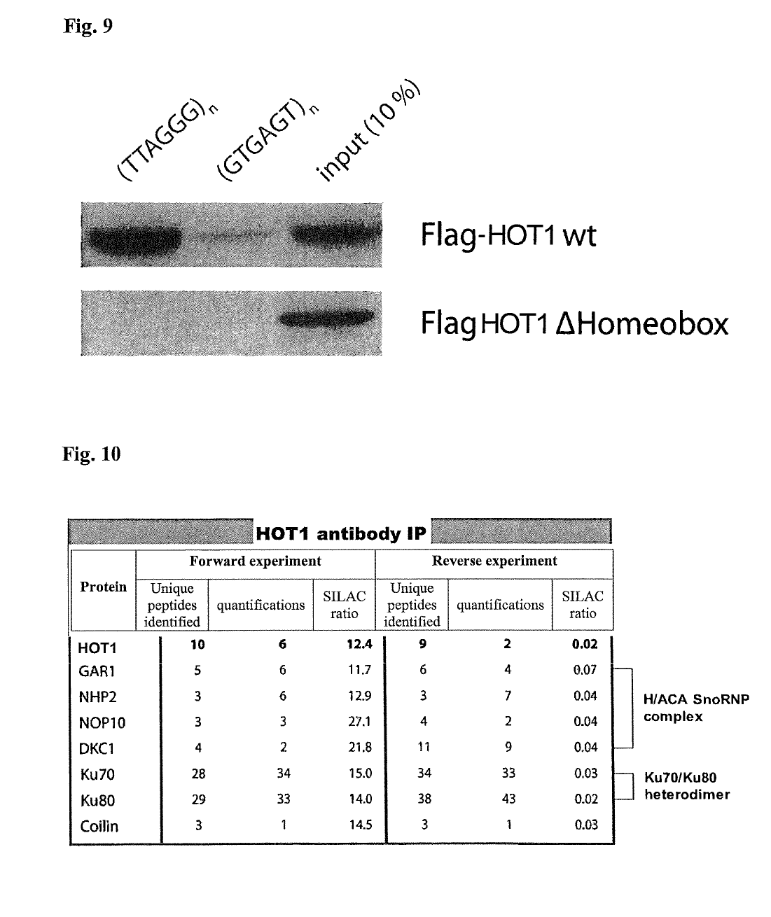

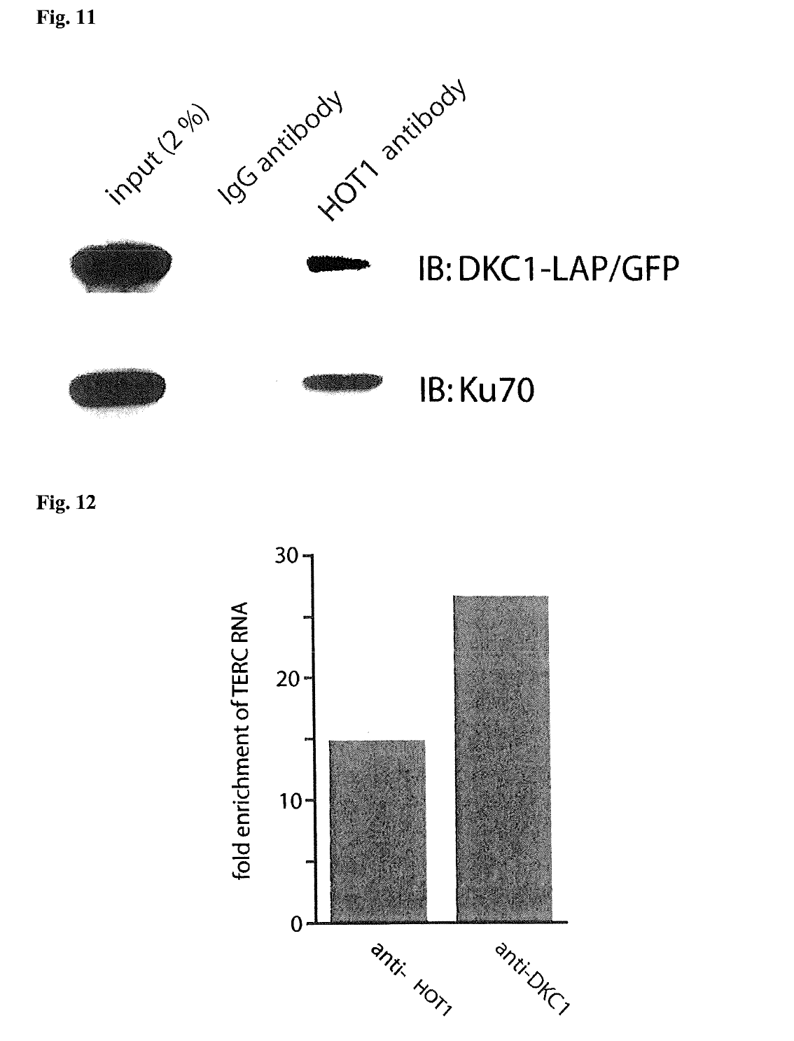

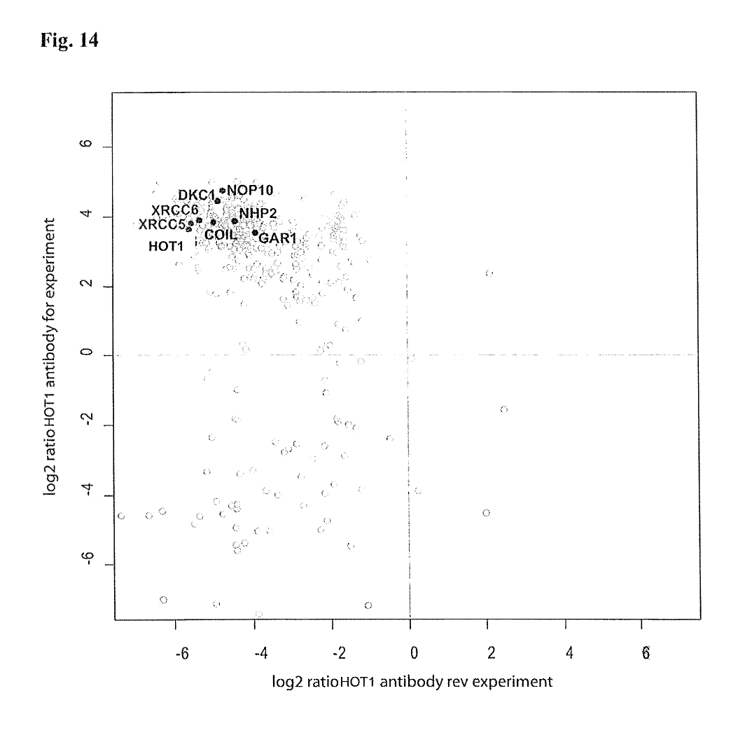

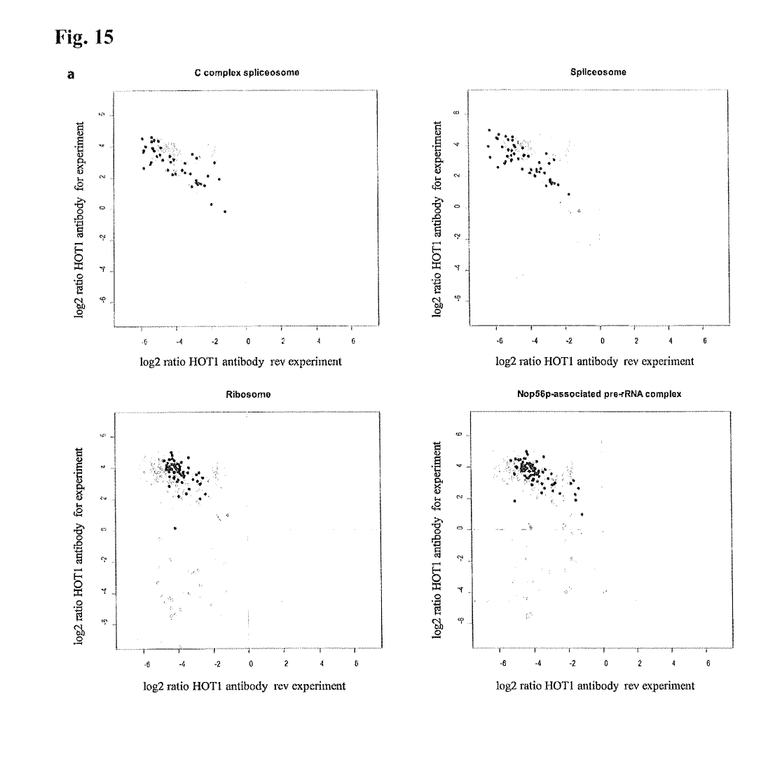

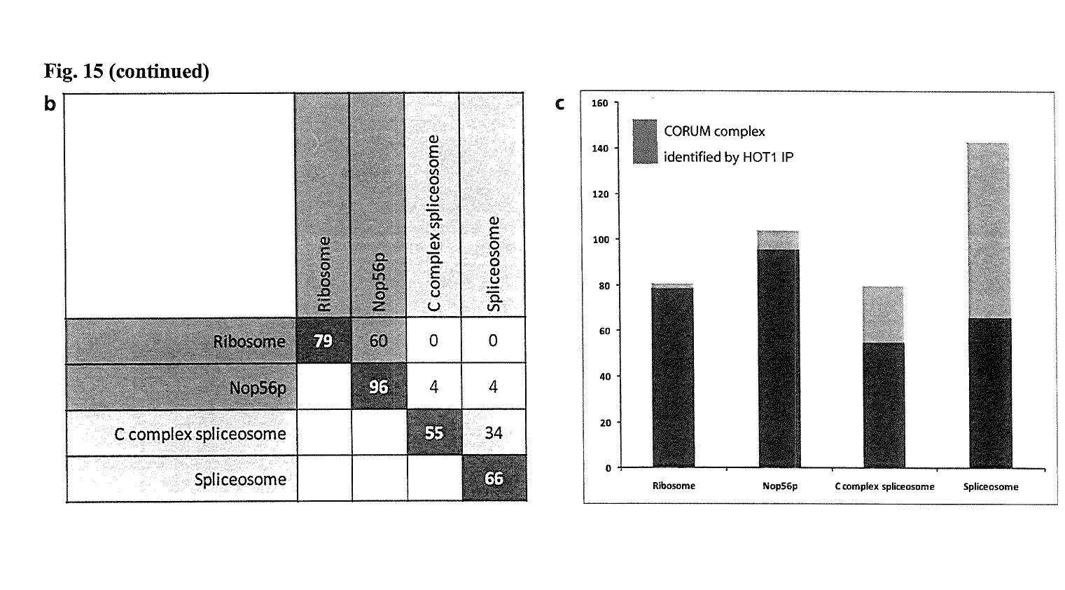

To test whether HOT1-dependent telomere elongation requires DNA binding, the inventors investigated the effect of a mutant of HOT1, in which the putative DNA binding region, the homeobox domain was deleted. In vitro binding studies with recombinant HOT1ΔHomeobox demonstrated that the deletion of this domain indeed completely abolished binding to telomeric TTAGGG (SEQ ID NO: 7) repeats (FIG. 9). To examine functional consequences of DNA-binding deficient HOT1 on telomere length regulation, the inventors overexpressed Flag-HOT1ΔHomeobox in HeLa cells. In contrast to telomere elongation upon expression of full-length Flag-HOT1, expression of Flag-HOT1ΔHomeobox did not lead to a significant effect on telomere length in comparison to the control (FIG. 8). This lack of lengthening observed upon loss of telomere binding establishes that HOT1-telomere interaction is essential for telomere elongation. To further elucidate HOT1 function at telomeres, the inventors performed HOT1 immunoprecipitation (IP) assays, utilizing a HOT1 antibody combined with quantitative mass spectrometry (Vermeulen, M., et al. Curr. Opin. Biotechnol. 19, 331-337 (2008)). HOT1 was recovered with SILAC ratios indicative of specific binding to the antibody (FIG. 10). Remarkably, the inventors also identified several proteins relevant for telomere homeostasis: the four core components of box H/ACA snoRNPs (DKC1, GAR1, NHP2 and NOP10; part of the active telomerase RNP) and the Ku70-Ku80 heterodimer proteins. The inventors validated several of these interactions by co-IP experiments, confirming a physical interaction of HOT1 with components of the active telomerase complex (FIG. 11). In addition, Caja1 body proteins, notably the CB scaffolding and marker protein Coilin, were strongly enriched in the IP experiment (FIG. 10, FIG. 14, FIG. 15), indicating HOT1 functions in the Caja1 body.

The interaction of HOT 1 with the active telomerase complex components box H/ACA snoRNPs subunits raises the possibility that HOT1 binds to active telomerase. To substantiate this hypothesis, we performed RNA immunoprecipitation experiments with antibodies directed against HOT1 and observed a marked enrichment for TERC, the telomerase RNA component, in the HOT1 IP fraction (FIG. 12).

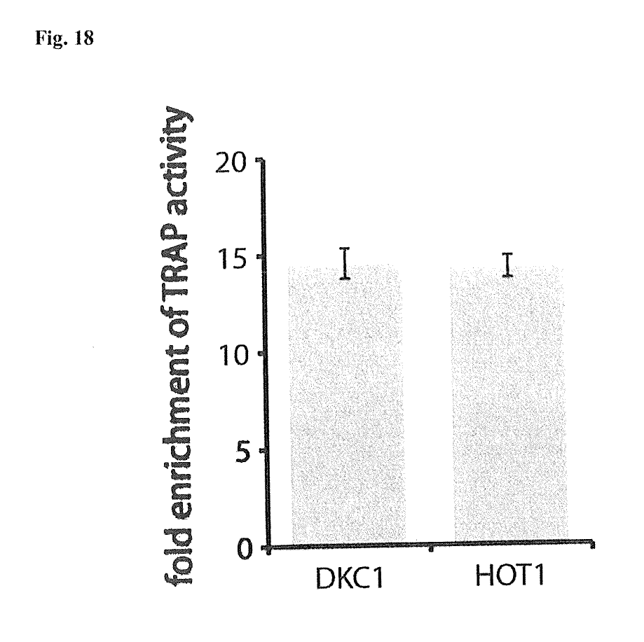

To address whether this signifies an association with the active telomerase complex we then tested whether HOT1 immunoprecipitates telomerase activity as well. Indeed we detected telomerase activity measured by the quantitative TRAP (telomere repeat amplification protocol) assay for immunoprecipitates obtained from IPs for both HOT1 and the positive control DKC1 (FIG. 18). We conclude that HOT1 physically interacts with the active telomerase complex.

To corroborate the physical interaction between HOT1 and Coilin we performed immunofluorescence (IF) stainings for HOT1 and Coilin in non-synchronized HeLa cells. After deconvolution and 3D reconstruction of the IF images the co-localization between both proteins was analyzed. In about 85% of all cells analyzed we observed colocalization of one to seven HOT1 foci with Caja1 bodies. Remarkably, HOT1 foci preferentially localized to the periphery of Coilin, reminiscent of previous findings on the association of telomerase RNA and telomeres with Caja1 bodies.

This further underscores the association of HOT1 with active telomerase and establishes HOT1 as the first telomere-binding protein that binds both telomerase and Caja1 body components.

Recently, WDR79 (also known as TCAB1) has been shown to be required for proper localization of CAB box containing small Caja1 body-specific RNPs (scaRNPs) to Caja1 bodies, including TERC (Tycowski, K. T., et al. Mol. Cell. 34, 47-57 (2009); Venteicher, A. S. et al. Science 323, 644-648 (2009)). Similar to HOT1, WDR79 acts as positive regulator of telomere length by ensuring proper TERC recruitment to Caja1 bodies (Venteicher, A. S. et al., supra). The telomere binding properties of HOT1 and its positive effects on telomere length regulation suggest that HOT1 acts downstream of TCAB likely by recruiting telomerase-containing Caja1 bodies to telomeres thus promoting telomerase association with telomeres, perhaps through additional interactions involving telomerase and TPP1. The fact that overexpression of HOT1 increases telomere length suggests that HOT1, like TERT and TERC, is also limiting. Therefore, the shelterin complex and the levels of TERT, TERC and HOT1 appear to act cooperatively to define a mean telomere length in the cell.

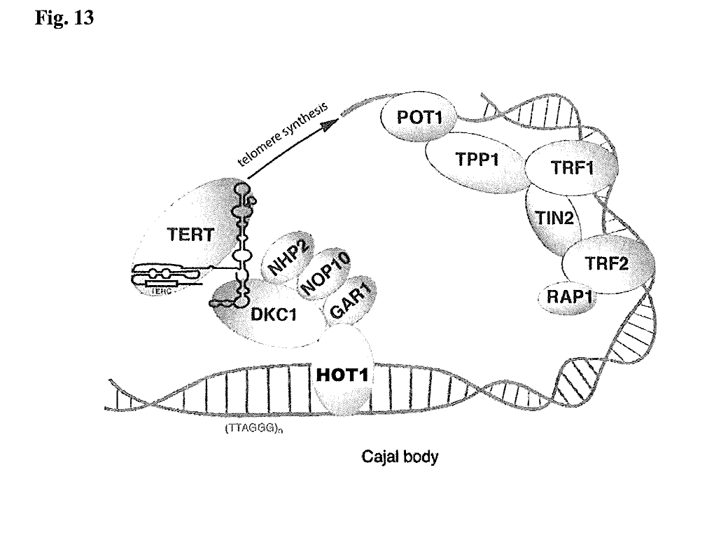

In conclusion, the inventors have identified HOT1 as the first telomere-repeat binding protein that associates both with Caja1 bodies and with telomerase and promotes telomere lengthening. Thus, HOT1 is a putative telomere and/or telomerase recruitment factor that may bridge telomerase to telomeres through binding of both telomeric DNA and the telomerase RNP (FIG. 13).

### Example 3

### Classifying a Cancer as a Telomerase-Negative Cancer

In this assay cells were fixed with 3% paraformaldehyd solution (in 1×PBS supplemented with 5 mM EGTA and 1 mM MgCl₂) for 10 min at room temperature. Cells were then washed twice with a 1×PBS solution containing 30 mM glycine. A permeabilization step with 1×PBS with 0.5% TritonX-100 at 4° C. for 5 min was followed by two additional washes with the 1×PBS+30 mM glycine solution. Cells were then blocked for 15 min at room temperature in blocking solution (1×PBS containing 0.2% fish skin gelatine) PML and HOT1 were marked by staining with primary antibodies against PML (as an APB marker; mouse monoclonal anti-PML PG-M3 antibody, Santa Cruz sc-966; 1:500 dilution) and HOT1 (MPI-CBG Antibody Facility; 1:1000 dilution) in blocking solution for 1 h at room temperature. After three washes for 3 min each with blocking solution secondary antibodies (goat-anti-mouse-Alexa488 and goat-anti-rabbit-Alexa562, Invitrogen; both at 1:500 dilution) were applied in blocking solution for 30 min at room temperature. After three additional washes with blocking solution cells were mounted in Antifade Gold mounting medium counting DAPI as a chromosome stain (Invitrogen). Fluorescent images were taken and within the nuclear area co-localization of HOT1 foci with PML bodies was analyzed.

### LIST OF REFERENCES

CN 101555486 (A)
CN101555484 (A)
Chen, S. et al. Isolation and functional analysis of human HMBOX1, a homeobox containing protein with transcriptional repressor activity. Cytogenet. Genome Res. 114, 131-136 (2006).
van Steensel, B. & de Lange, T. Control of telomere length by the human telomeric protein TRF1. Nature 385: 740-743 (1997).
Déjardin, J. & Kingston, R. E. Purification of proteins associated with specific genomic Loci. Cell 136, 175-186 (2009).
Ong, S. E. et al. Stable isotope labeling by amino acids in cell culture, SILAC, as a simple and accurate approach to expression proteomics. Mol. Cell. Proteomics 1, 376-386 (2002).
Butter, F., Kappei, D., Buchholz, F., Vermeulen, M. & Mann, M. A domesticated transposon mediates the effects of a single-nucleotide polymorphism responsible for enhanced muscle growth. EMBO Rep. 11, 305-311 (2010).
Mittler, G., Butter, F. & Mann, M. A SILAC-based DNA-protein interaction screen that identifies candidate binding proteins to functional DNA elements. Genome Res. 19, 284-293 (2009).
Kittler, R., Heninger, A. K., Franke, K., Habermann, B. & Buchholz, F. Production of endoribonuclease-prepared short-interfering RNAs for gene silencing in mammalian cells. Nat. Methods 2, 779-784 (2005).
Londoño-Vallejo, J. A., DerSarkissian, H., Cazes, L & Thomas, G. Differences in telomere length between homologous chromosomes in humans. Nucleic Acids Res 29, 3164-3171 (2001).
Vermeulen, M., Hubner, N. C. & Mann, M. High confidence determination of specific protein-protein interactions using quantitative mass spectrometry. Curr. Opin. Biotechnol. 19, 331-337 (2008).
Tycowski, K. T., Shu, M. D., Kukoyi, A. & Steitz, J. A. A conserved WD40 protein binds the Caja1 body localization signal of scaRNP particles. Mol. Cell. 34, 47-57 (2009).
Venteicher, A. S. et al. A human telomerase holoenzyme required for Caja1 body localization and telomere synthesis. Science 323, 644-648 (2009).
Li, M. Z. & Elledge, S. J. Harnessing homologous recombination in vitro to generate recombinant DNA via SLIC. Nat. Methods 4, 251-256 (2007).
Henschel, A., Buchholz, F. & Habermann, B. DEQOR: a web-based tool for the design and quality control of siRNAs. Nucleic Acids Res. 32, W113-120 (2004).
Poser, I. et al. BAC TransgeneOmics: a high-throughput method for exploration of protein function in mammals. Nat. Methods 5, 409-415.

1. A method of treating a disorder associated with abnormal telomerase activity and/or abnormal telomere length in comparison to healthy subjects in a subject, comprising administering a polynucleotide encoding a polypeptide comprising an amino acid sequence having at least 70% homology to the amino acid sequence shown in SEQ ID NO: 2, wherein the polypeptide is capable of directly binding to telomere repeats and to the telomerase complex, or a fragment of said polynucleotide;
wherein direct binding to telomere repeats is determined by in vitro telomere pulldown, and wherein direct binding to the telomerase complex is determined by RNA immunoprecipitation and quantitative real-time PCR.

2. The method of claim 1, wherein the disorder associated with abnormal telomerase activity and/or abnormal telomere length is a premature-aging syndrome,
wherein said polynucleotide is used in order to increase the expression of said polypeptide in the cells of said subject.

3. The method of claim 1, wherein the disorder associated with abnormal telomerase activity and/or abnormal telomere length is dyskeratosis congenita, aplastic anemia, or idiopathic pulmonary fibrosis.

4. The method of claim 1, wherein the disorder associated with abnormal telomerase activity and/or abnormal telomere length is cancer, selected from the group of cancers consisting of lung cancer, breast cancer, colorectal cancer, pancreatic cancer, ovarian cancer, prostate cancer, bladder cancer, uterine cancer, liver cancer, kidney cancer, endometrial cancer, thyroid cancer, oral or pharyngeal cancer, esopharyngeal cancer, brain cancer, skin melanoma, leukaemia, fibrosarcoma, and non-Hodgkin lymphoma, wherein the polynucleotide or fragment thereof is used to reduce the expression of said polypeptide in the cancer cells of said subject.

5. The method of claim 4, wherein the fragment is an esiRNA.

6. A method of treating a premature-aging syndrome, comprising administering a polypeptide comprising an amino acid sequence having at least 70% homology to the amino acid sequence shown in SEQ ID NO: 2, wherein the polypeptide is capable of directly binding to telomere repeats and to the telomerase complex,
wherein direct binding to telomere repeats is determined by in vitro telomere pulldown, and wherein direct binding to the telomerase complex is determined by RNA immunoprecipitation and quantitative real-time PCR.

7. The method of claim 6, wherein the premature-aging syndrome is selected from the group consisting of dyskeratosis congenita, Werner syndrome, Cockayne syndrome, Bloom syndrome, ataxia telangiectasia, ataxia telangiectasia-like disorder, Nijmegen breakage syndrome, Cri du chat syndrome, Fanconi anemia, xeroderma pigmento sum, Hoyeraal-Hreidars son syndrome, Li-Fraumeni syndrome, Niedernhofer syndrome, and Hutchinson-Gilford progeria syndrome.

8. A method of treating a disorder associated with abnormal telomerase activity and/or abnormal telomere length, wherein the disorder is selected from dyskeratosis congenita, aplastic anemia, and idiopathic pulmonary fibrosis, comprising administering a polypeptide comprising an amino acid sequence having at least 70% homology to the amino acid sequence shown in SEQ ID NO: 2, wherein the polypeptide is capable of directly binding to telomere repeats and to the telomerase complex;
wherein direct binding to telomere repeats is determined by in vitro telomere pulldown, and wherein direct binding to the telomerase complex is determined by RNA immunoprecipitation and quantitative real-time PCR.

9. The method of claim 6, 7 or 8, wherein the polypeptide consists of an amino acid sequence having at least 70% homology to the amino acid sequence shown in SEQ ID NO: 2.

10. The method of claim 6, 7 or 8, wherein the polypeptide comprises the amino acid sequence shown in SEQ ID NO: 2.

11. A method of identifying an agent capable of decreasing telomerase activity, comprising the steps of
(i) contacting a candidate agent with a polypeptide comprising an amino acid sequence having at least 70% homology to the amino acid sequence shown in SEQ ID NO: 2,
wherein the polypeptide is capable of directly binding to telomere repeats and to the telomerase complex;
wherein direct binding to telomere repeats is determined by in vitro telomere pulldown, and/or
wherein direct binding to the telomerase complex is determined by RNA immunoprecipitation and quantitative real-time PCR; and
(ii) determining if the polypeptide subjected to step (i) is capable of
(a) binding to telomere repeats, and/or
(b) binding to the telomerase complex;
wherein a reduced binding determined in step (ii) (a) and/or step (ii) (b) compared to that of a corresponding polypeptide not subjected to step (i) is indicative of an agent capable of decreasing the telomerase activity.

12. A method of classifying a cancer as a telomerase-negative cancer, comprising the step of
determining the co-localisation of promyelocytic leukaemia (PML) bodies and a polypeptide comprising an amino acid sequence having at least 70% homology to the amino acid sequence shown in SEQ ID NO: 2, wherein the polypeptide is capable of directly binding to telomere repeats and to the telomerase complex,
wherein direct binding to telomere repeats is determined by in vitro telomere pulldown, and
wherein direct binding to the telomerase complex is determined by RNA immunoprecipitation and quantitative real-time PCR;
in a cell comprised in a specimen of the cancer to be classified;
wherein a co-localisation is indicative of a telomerase-negative cancer.

13. The method of claim 12, wherein the co-localisation is determined by immunofluorescence.

14. The method of claim 2, wherein the premature-aging syndrome is selected from the group consisting of dyskeratosis congenita, Werner syndrome, Cockayne syndrome, Bloom syndrome, ataxia telangiectasia, ataxia telangiectasia-like disorder, Nijmegen breakage syndrome, Cri du chat syndrome, Fanconi anemia, xeroderma pigmentosum, Hoyeraal-Hreidarsson syndrome, Li-Fraumeni syndrome, Niedernhofer syndrome, and Hutchinson-Gilford progeria syndrome.

15. The method of claim 6, wherein the polypeptide consists of the amino acid sequence shown in SEQ ID NO: 2.

16. The method of claim 7, wherein the polypeptide consists of an amino acid sequence having at least 70% homology to the amino acid sequence shown in SEQ ID NO: 2.

17. The method of claim 7, wherein the polypeptide comprises the amino acid sequence shown in SEQ ID NO: 2.

18. The method of claim 7, wherein the polypeptide consists of the amino acid sequence shown in SEQ ID NO: 2.

19. The method of claim 8, wherein the polypeptide consists of an amino acid sequence having at least 70% homology to the amino acid sequence shown in SEQ ID NO: 2.

20. The method of claim 8, wherein the polypeptide comprises the amino acid sequence shown in SEQ ID NO: 2.

21. The method of claim 8, wherein the polypeptide consists of the amino acid sequence shown in SEQ ID NO: 2.
